(19) United States
(12) Patent Application Publication
(10) Pub. No.: US 2021/0371881 A1
(43) Pub. Date: Dec. 2, 2021

(54) DEVICES AND METHODS FOR TRANSFECTION

(71) Applicant: TransCytos, LLC, Sturbridge, MA (US)

(72) Inventors: Otto J. Prohaska, Sturbridge, MA (US); Rainer Fasching, Sturbridge, MA (US); Fethi I. Olcaytug, Sturbridge, MA (US); Theresa L. O'Keefe, Sturbridge, MA (US); Srinivas Shankara, Sturbridge, MA (US); Arumugham Raghunathan, Sturbridge, MA (US); David Bonneau, Sturbridge, MA (US)

(73) Assignee: TransCytos, LLC, Sturbridge, MA (US)

(21) Appl. No.: 17/331,166

(22) Filed: May 26, 2021

Related U.S. Application Data
(60) Provisional application No. 63/030,025, filed on May 26, 2020.

(51) Int. Cl. C12N 15/87; B01L 3/00

(52) U.S. Cl. CPC C12N 15/87; B01L 3/502761; B01L 3/502746; B01L 3/50273; B01L 2200/0647; B01L 2400/086; B01L 2400/0487; B01L 2300/16; B01L 2400/088

(57) ABSTRACT

Disclosed herein are methods, assemblies, systems, kits and devices for introducing molecules or compositions into cells or cell-like bodies. An assembly for introducing molecules in a solution into cells or cell-like bodies comprises a rigid container having a first inner diameter or cross-sectional area at a proximal end thereof and inner and outer walls extending between a distal and proximal end, a plunger insertable into the container at the proximal end, and at least one constriction of only the inner wall proximal to the distal end or at least one constriction of the inner and the outer walls proximal to the distal end, wherein the at least one constriction has a second inner diameter or cross-sectional area that is smaller than the container first inner diameter or cross-sectional area and the plunger is axially movable along the container.

### RELATED APPLICATIONS

This application claims priority to U.S. Provisional Application No. 63/030,025, filed May 26, 2020. The contents of the foregoing priority application are incorporated by reference herein in its entirety.

### BACKGROUND

Transfection—the introduction of a molecule or composition, e.g., DNA, RNA or proteins, into living cells—is a fundamental and essential genetic engineering process in biomedical research, drug development, and gene therapy. It is used by scientists throughout the world to study diseases such as cancer, obesity, heart diseases, diabetes, arthritis, substance abuse, Parkinson's, and Alzheimer's, as well as topics related to anxiety and aging. Transfection enables the production of recombinant human proteins such as hormones (e.g. insulin), antibodies and vaccines, and enables disease therapies based on treatment with peptides, proteins, DNA and RNA.

While the transfection process itself was discovered decades ago, it has until now been mostly limited to use with certain cell types. Existing technologies can be broadly classified into three groups: chemical, biological and physical. Chemical methods, such as cationic lipid transfection, calcium phosphate transfection, DEAE-dextran transfection and delivery by other cationic polymers (e.g., polybrene, PEI, dendrimers), use carrier molecules to neutralize or impart a positive charge to negatively charged nucleic acids being transfected. Biological methods rely on genetically engineered viruses to transfer genes into cells (also known as transduction). Physical methods, such as electroporation, biolistic particle delivery (particle bombardment), direct microinjection and laser-mediated transfection (phototransfection), directly deliver molecules into the cytoplasm or nucleus of a cell. No one method can be applied to all cell types or used to deliver all types of molecules. Moreover, such techniques represent a considerable bottleneck in research and disease treatment as they result in low efficiency (number of transfected cells), low viability (number of cells surviving), high variability, cellular toxicity, and the inability to introduce material into many of the most important cell types relevant to major diseases including immune cells and stem cells.

### SUMMARY

Disclosed herein are devices, systems, kits and methods for performing transfections.

There remains a need for a transfection system and method having high efficiency (high number of cells transfected), high viability (high number of cells surviving), low variability, low cell toxicity, fast cell recovery and ability to transform a multitude of cell sizes and types.

In one aspect of at least one embodiment, an assembly for introducing molecules in a solution into cells or cell-like bodies is provided including a rigid container including a first inner diameter or cross-sectional area at a proximal end thereof and inner and outer walls extending between a distal and proximal end; a plunger insertable into the container at the proximal end; and at least one constriction of only the inner wall proximal to the distal end or at least one constriction of the inner and the outer walls proximal to the distal end; wherein the at least one constriction has a second inner diameter or cross-sectional area that is smaller than the container first inner diameter or cross-sectional area and the plunger is axially movable along the container.

In some embodiments of the assembly, the container includes ripples that protrude away from an interior wall of the container.

In some embodiments of the assembly, the constriction has a diameter that is 1.2 to 100 times larger than a diameter of the cells or cell-like bodies.

In some embodiments of the assembly, the constriction has a diameter that is 2 to 10 times larger than a diameter of the cells or cell-like bodies.

In some embodiments of the assembly, the plunger includes a rod having a distal and proximal end wherein the distal end of the plunger is a conical or cylindrical tip and the proximal end of the plunger is configured to attach the plunger to a motorized arm.

In some embodiments of the assembly, an average roughness of an inner wall of the container is 10 nm-1 μm.

In some embodiments of the assembly, the roughness is created by adsorbing cell fragments to the inner wall of the container.

In another aspect, an assembly for introducing molecules in a solution into cells or cell-like bodies is provided including a flexible container including a first inner diameter or cross-sectional area and a first and second end; at least one constriction formed by compressing at least one section of the flexible container; and optionally, a removable plunger positioned at least at one of the first or second ends or removable plungers positioned at each of the first and second ends; wherein the at least one constriction section has a second inner diameter or cross-sectional area that is smaller than the container first inner diameter or cross-sectional area.

In some embodiments of the assembly, the plungers are axially movable along the container or are replaced by stationary caps at the ends of the container.

In some embodiments of the assembly, the at least one constriction section is formed by at least one movable wedge or at least one movable roller.

In some embodiments of the assemblies described above, the container includes multiple constrictions. Each of the multiple constrictions can have the same inner diameter or cross-sectional area, differing inner diameters or cross-sectional areas or combinations thereof. In some embodiments, the constriction forms a flow path having a length of a 0.2-10 mm. In some embodiments of the assemblies described above, the container includes a removable insert having multiple constrictions.

In one aspect of at least one embodiment, an assembly for introducing molecules in a solution into cells or cell-like bodies is provided including a rigid container having inner and outer walls extending between a distal and proximal end, the outer and inner walls narrow to form a constriction in a central portion of the container between the distal and proximal ends, a plunger movably disposed in the container near the proximal end, and the constriction having a diameter that is 1.2 to 100 times larger than a diameter of the cells or cell-like bodies.

In another aspect of at least one embodiment, a microfluidic device for introducing molecules in a solution into cells or cell-like bodies is provided including a flexible container comprising a first inner diameter or cross-sectional area and a first and second end, at least one movable wedge movable along the container that compresses the container to form a constriction, and stationary caps fixed at the first and second ends of the container, wherein the at least one constriction has a second inner diameter or cross-sectional area that is smaller than the container first inner diameter or cross-sectional area, wherein the constriction has a diameter that is 1.2 to 100 times larger than a diameter of the cells or cell-like bodies.

In another aspect of at least one embodiment, a microfluidic device for introducing molecules in a solution into cells or cell-like bodies is provided including at least one channel having a first inner diameter or cross-sectional area; at least one constriction section contiguous with the channel; and at least one structure configured to at least partially enter the channel; wherein the at least one constriction section has a second inner diameter or cross-sectional area that is smaller than the channel first inner diameter or cross-sectional area.

In some embodiments of the microfluidic device, the at least one structure is a plunger or a flexible sheet.

In some embodiments of the microfluidic device, the device includes multiple channels. In some embodiments of the microfluidic device, the channel or channels include multiple constrictions. Each of the multiple constrictions can have the same inner diameter or cross-sectional area, differing inner diameters or cross-sectional areas or combinations thereof.

In at least some embodiments of the assemblies and microfluidic devices described herein (whether alone or as part of a system as described below), the inner diameter of the constriction section is about 1.2 to 100 times larger than the diameter of the cells or cell-like bodies being transfected and the inner cross-sectional area of the constriction section is about 1.5 to 10,000 times larger than the cross-sectional area of the cells or cell-like bodies being transfected.

In another aspect of at least one embodiment, a system for introducing molecules in a solution into cells or cell-like bodies is provided including an instrument including at least one arm attached to a motor, the motor configured to axially move the at least one arm; and at least one assembly including a rigid container including a first inner diameter or cross-sectional area and inner and outer walls extending between a distal and proximal end; a plunger insertable into the container at the proximal end; and at least one constriction of only the inner wall at the distal end or at least one constriction of the inner and the outer walls proximal to the distal end; wherein the at least one constriction has a second inner diameter or cross-sectional area that is smaller than the container first inner diameter or cross-sectional area and the plunger is axially movable along the container.

In some embodiments of the system, the plunger is attached to the at least one arm. In some embodiments, multiple plungers are attached to the arm or multiple plungers are attached to multiple arms.

In another aspect of at least one embodiment, a system for introducing molecules in a solution into cells or cell-like bodies is provided including an instrument including at least one arm attached to a motor, the motor configured to axially move the at least one arm; and at least one assembly including a flexible container including a first inner diameter or cross-sectional area and a first and second end; at least one constriction formed by compressing at least one section of the flexible container; and optionally, a removable plunger positioned at least at one of the first or second ends or removable plungers positioned at each of the first and second ends; wherein the at least one constriction has a second inner diameter or cross-sectional area that is smaller than the container first inner diameter or cross-sectional area

In some embodiments of the system, the at least one constriction section is formed by at least one movable wedge or at least one movable roller. In certain embodiments, the wedge or roller is attached to the at least one arm. In other embodiments, multiple wedges or rollers are attached to the arm or multiple wedges or rollers are attached to multiple arms.

In some embodiments of the systems described above, the system includes multiple assemblies.

In another aspect of at least one embodiment, a system for introducing molecules in a solution into cells or cell-like bodies is provided including an instrument including at least one arm attached to a motor, the motor configured to axially move the at least one arm; and at least one microfluidic device including at least one channel having a first inner diameter or cross-sectional area; at least one constriction section contiguous with the channel; and at least one structure configured to at least partially enter the channel; wherein the at least one constriction section has a second inner diameter or cross-sectional area that is smaller than the channel first inner diameter or cross-sectional area and the at least one structure is at least one plunger attached to the at least one arm.

In some embodiments of the system, multiple plungers are attached to the arm or multiple plungers are attached to multiple arms.

In another aspect of at least one embodiment, a system for introducing molecules in a solution into cells or cell-like bodies is provided including an instrument including at least one piezoelectric stack; and at least one microfluidic device including at least one channel having a first inner diameter or cross-sectional area; at least one constriction section contiguous with the channel; and at least one structure configured to at least partially enter the channel; wherein the at least one constriction section has a second inner diameter or cross-sectional area that is smaller than the channel first inner diameter or cross-sectional area and the at least one structure is at least one flexible sheet in contact with the at least one piezoelectric stack.

In some embodiments of the system, multiple flexible sheets are in contact with the piezoelectric stack or multiple flexible sheets are in contact with multiple piezoelectric stacks.

In some embodiments of any of the systems described above, the channel or channels include multiple constrictions. Each of the multiple constrictions can have the same inner diameter or cross-sectional area or differing inner diameters or cross-sectional areas.

In some embodiments of any of the systems described above, the system further includes at least one optical sensor.

In another aspect of at least one embodiment, a kit for introducing molecules in a solution into cells or cell-like bodies is provided including at least one assembly including a rigid container including a first inner diameter or cross-sectional area and inner and outer walls extending between a distal and proximal end; a plunger insertable into the container at the proximal end; at least one constriction of only the inner wall at the distal end or at least one constriction of the inner and the outer walls proximal to the distal end; and at least one transfection solution contained within the at least one container and/or at least one transfection solution in at least one separate vial; wherein the at least one constriction has a second inner diameter or cross-sectional area that is smaller than the container first inner diameter or cross-sectional area and the plunger is axially movable along the container.

In another aspect of at least one embodiment, a kit for introducing molecules in a solution into cells or cell-like bodies is provided including at least one assembly including a flexible container including a first inner diameter or cross-sectional area and a first and second end; at least one constriction formed by compressing at least one section of the flexible container; optionally, a removable plunger positioned at least at one of the first or second ends or removable plungers positioned at each of the first and second ends; and at least one transfection solution contained within the at least one container and/or at least one transfection solution in at least one separate vial; wherein the at least one constriction section has a second inner diameter or cross-sectional area that is smaller than the container first inner diameter or cross-sectional area.

In another aspect of at least one embodiment, a kit for introducing molecules in a solution into cells or cell-like bodies is provided including at least one microfluidic device including at least one channel having a first inner diameter or cross-sectional area; at least one constriction section contiguous with the channel; at least one structure configured to at least partially enter the at least one channel; and at least one transfection solution contained within the at least one channel and/or at least one transfection solution in at least one separate vial; wherein the at least one constriction section has a second inner diameter or cross-sectional area that is smaller than the channel first inner diameter or cross-sectional area.

In another aspect of at least one embodiment, a method for introducing molecules from a solution into cells or cell-like bodies is provided including a) providing a solution containing cells or cell-like bodies and transfection material, the solution in contact with at least one movable structure; and b) passing the sample solution through at least one constriction at least one time by moving the movable structure wherein the at least one constriction has a diameter that is 1.2 to 100 times larger than a diameter of the cells or cell-like bodies.

In some embodiments of the method, the movable structure is a plunger insertable into a rigid container and axially movable along the container. The container includes a first inner diameter or cross-sectional area and inner and outer walls extending between a distal and proximal end and at least one constriction of only the inner wall at the distal end or at least one constriction of the inner and the outer walls proximal to the distal end; wherein the at least one constriction has a second inner diameter or cross-sectional area that is smaller than the container first inner diameter or cross-sectional area. Thus, the method is performed using the appropriate assemblies and systems described above.

In some embodiments of the method, the movable structure is a flexible container compressible by at least one movable wedge or roller. The flexible container includes inner surfaces, a first inner diameter or cross-sectional area and a first and second end and, optionally, a removable plunger positioned at least at one of the first or second ends or removable plungers positioned at each of the first and second ends; wherein the at least one constriction formed by compressing the flexible container has a second inner diameter or cross-sectional area that is smaller than the container first inner diameter or cross-sectional area. Thus, the method is performed using the appropriate assemblies and systems described above. In some embodiments, at least one of a shape, size and position of the movable wedge or roller is selected to adjust the size of the constriction.

In some embodiments of the method, the movable structure is a plunger at least partially insertable into a channel of a microfluidic device. The microfluidic device includes at least one channel having a first inner diameter or cross-sectional area and at least one constriction section contiguous with the channel; wherein the at least one constriction section has a second inner diameter or cross-sectional area that is smaller than the channel first inner diameter or cross-sectional area. Thus, the method is performed using the appropriate assemblies and systems described above.

In some embodiments of the method, the movable structure is a flexible sheet at least partially insertable into a channel of a microfluidic device. The microfluidic device includes at least one channel having a first inner diameter or cross-sectional area and at least one constriction section contiguous with the channel; wherein the at least one constriction section has a second inner diameter or cross-sectional area that is smaller than the channel first inner diameter or cross-sectional area. Thus, the method is performed using the appropriate assemblies and systems described above.

In another aspect of at least one embodiment, a method for introducing molecules from a solution into cells or cell-like bodies is provided including a) providing a solution containing cells or cell-like bodies and transfection material; b) loading the solution into at least one rigid container including a first inner diameter or cross-sectional area, at least one constriction having a second inner diameter or cross-sectional area that is smaller than the container first inner diameter or cross-sectional area and a plunger, wherein the solution is in contact with the plunger; and c) moving the plunger axially within the container to pass the solution through the at least one constriction at least one time.

In another aspect of at least one embodiment, a method for introducing molecules from a solution into cells or cell-like bodies is provided including a) providing a solution containing cells or cell-like bodies and transfection material; b) loading the solution into at least one flexible container including inner surfaces, a first inner diameter or cross-sectional area and a first and second end, at least one constriction formed by compressing at least one section of the flexible container, the constriction having a second inner diameter or cross-sectional area that is smaller than the container first inner diameter or cross-sectional area and optionally, a removable plunger positioned at least at one of the first or second ends or removable plungers at each of the first and second ends of the container, wherein the solution is in contact with the inner surfaces of the flexible container; and c) moving at least one wedge or roller axial along the container to pass the solution through the at least one constriction at least one time.

In another aspect of at least one embodiment, a method for introducing molecules in a solution into cells or cell-like bodies is provided including a) providing a solution containing cells or cell-like bodies and transfection material; b) loading the solution into at least one microfluidic device including at least one channel having a first inner diameter or cross-sectional area, at least one constriction section contiguous with the channel, the constriction section having a second inner diameter or cross-sectional area that is smaller than the channel first inner diameter or cross-sectional area and at least one structure configured to at least partially enter the channel, wherein the sample is in contact with the structure; and c) moving the structure within the channel to pass the solution through the at least one constriction at least one time. In some embodiments, the structure is at least one plunger or at least one flexible sheet.

In some embodiments of the methods, the transfection material includes genetic material, peptides, proteins, carbohydrates, lipids, inorganic compounds, synthetic polymers, drugs, pharmaceutical compositions or mixtures thereof. In certain embodiments, the transfection material is proteins that are antibodies or fragments thereof. In other embodiments, the transfection material is genetic material that is an expression vector encoding antibodies, antibody fragments or chimeric antigen receptors (CARs). In yet other embodiments, the transfection material is a mixture of protein and genetic material, such as ribonucleoproteins (RNP) including gene editing components or gene editing complexes. In certain embodiments, the gene editing components or gene editing complexes include CRISPR components, such as a Cas protein or Cpf1 protein and guide RNA (gRNA), donor DNA or a CRISPR RNA (crRNA) and a trans-activating crRNA (tracrRNA). In yet other embodiments, the gene editing components or gene editing complexes include a TALEN protein, a zinc finger nuclease (ZFN), a mega nuclease or a Cre recombinase.

In some embodiments of the methods, the cells include prokaryotic cells or eukaryotic cells. In certain embodiments, the prokaryotic cells are bacteria, cyanobacteria or archaea. In certain embodiments, the eukaryotic cells are animal cells, plant cells, yeast, protists or fungi. In some embodiments of the methods, the cell-like bodies include exosomes, vesicles, organelles, membrane-bound sub-cellular vesicles, cell-derived or synthetically-derived membrane bound vesicles or cell-derived or synthetically-derived sub-cellular vesicles.

In yet other embodiments, the eukaryotic cells are epithelial cells, hematopoietic cells, stem cells, spleen cells, kidney cells, pancreas cells, liver cells, neuron cells, glial cells, muscle cells, heart cells, lung cells, ocular cells, bone marrow cells, gametes (oocytes and sperm cells), fetal cord blood cells, progenitor cells, tumor cells, peripheral blood mononuclear cells, immune cells including leukocyte cells, lymphocyte cells, T cells, B cells, natural killer (NK) cells, dendritic cells (DC), natural killer T (NKT) cells, mast cells, monocytes, macrophages, basophils, eosinophils or neutrophils. In still other embodiments, the eukaryotic cells are NIH 3T3 cells, algae, CHO cells, Cos-7 cells, epithelial cells, HEK293 cells, HeLa cells, HepG2 cells, HT-29 cells, B cells, human embryonic stem cells, HUVEC, Jurkat cells, K562 cells, MCF7 cells, MDCK cells, mouse embryonic stem cells, mesenchymal stem cells, PBMCs, PC12 cells, primary astrocytes, rat whole blood cells, rat dorsal root ganglion cells, red blood cells, rat neural stem cells, SF9 cells, SH-SY5Y cells, spleenocytes, U266 cells, U87-human glioblastoma cells, P. pastoris cells, S. cerevisiae cells or human oocytes. In certain embodiments, the immune cells are human T cells.

In some embodiments of the methods, the sample solution is passed through the constriction more than one time. In certain embodiments, the sample solution is passed through the constriction about 1-100 times, preferably about 30 times. In other embodiments, the sample solution is passed through the constriction about 10-90 times. In other embodiments, the sample solution is passed through the constriction about 15-50 times. In some embodiments, the sample solution is passed through the constriction about 15 times. In some embodiments, the sample solution is passed through the constriction about 20 times.

In some embodiments of the methods, the sample solution passes through the constriction at an average flow rate of about 10 μl/sec to about 1000 μl/sec.

In another aspect of at least one embodiment, a method for protecting a subject against an infectious agent is provided including: a) optionally, isolating cells from a mammal; b) providing autologous cells, allogenic cells or cell-like bodies; c) mixing the cells or cell-like bodies with a solution containing an expression vector encoding an antibody or an antibody fragment that binds to the infectious agent or to a toxic substance produced by the infectious agent to form a sample solution; d) loading the sample solution into at least one rigid container according to the assemblies described herein, wherein the sample is in contact with the plunger; e) moving the plunger axially within the container to pass the sample solution through the at least one constriction at least one time to transfect the cells or cell-like bodies; f) optionally, growing the cells ex vivo to increase the number of cells; and g) infusing the subject with said transfected cells or cell-like bodies.

In another aspect of at least one embodiment, a method for protecting a subject against an infectious agent is provided including: a) providing autologous cells, allogenic cells or cell-like bodies; b) mixing the cells or cell-like bodies with a solution containing an expression vector encoding an antibody or an antibody fragment that binds to the infectious agent or to a toxic substance produced by the infectious agent to form a sample solution; c) loading the sample solution into at least one rigid container according to the assemblies described herein, wherein the sample is in contact with the plunger; d) moving the plunger axially within the container to pass the sample solution through the at least one constriction at least one time to transfect the cells or cell-like bodies; and e) infusing the subject with said transfected cells or cell-like bodies. In some embodiments, the method includes isolating cells from a mammal. In some embodiments, the method includes growing the cells ex vivo to increase the number of cells.

In another aspect of at least one embodiment, a method for protecting a subject against an infectious agent is provided including: a) optionally, isolating cells from a mammal; b) providing autologous cells, allogenic cells or cell-like bodies; c) mixing the cells or cell-like bodies with a solution containing an expression vector encoding an antibody or an antibody fragment that binds to the infectious agent or to a toxic substance produced by the infectious agent to form a sample solution; d) loading the sample solution into at least one flexible container according to the assemblies described herein, wherein the sample is in contact with the inner surfaces of the flexible container; e) moving at least one wedge or roller axially along the container to pass the sample solution through the at least one constriction at least one time to transfect the cells or cell-like bodies; f) optionally, growing the cells ex vivo to increase the number of cells; and g) infusing the subject with said transfected cells or cell-like bodies.

In another aspect of at least one embodiment, a method for protecting a subject against an infectious agent is provided including: a) providing autologous cells, allogenic cells or cell-like bodies; b) mixing the cells or cell-like bodies with a solution containing an expression vector encoding an antibody or an antibody fragment that binds to the infectious agent or to a toxic substance produced by the infectious agent to form a sample solution; c) loading the sample solution into at least one flexible container according to the assemblies described herein, wherein the sample is in contact with the inner surfaces of the flexible container; d) moving at least one wedge or roller axially along the container to pass the sample solution through the at least one constriction at least one time to transfect the cells or cell-like bodies; and e) infusing the subject with said transfected cells or cell-like bodies. In some embodiments, the method includes isolating cells from a mammal. In some embodiments, the method includes growing the cells ex vivo to increase the number of cells.

In yet another aspect of at least one embodiment, a method for protecting a subject against an infectious agent is provided including: a) optionally, isolating cells from a mammal; b) providing autologous cells, allogenic cells or cell-like bodies; c) mixing the cells or cell-like bodies with a solution containing an expression vector encoding an antibody or an antibody fragment that binds to the infectious agent or to a toxic substance produced by the infectious agent to form a sample solution; d) loading said sample solution into at least one microfluidic device as described herein, wherein the sample is in contact with the structure; e) moving the structure within the at least one channel to pass the sample solution through the at least one constriction at least one time to transfect the cells or cell-like bodies; f) optionally, growing the cells ex vivo to increase the number of cells; and g) infusing the subject with said transfected cells or cell-like bodies.

In some embodiments of the methods for protecting a subject against an infectious agent, the infectious agent is bacteria, virus, fungi, parasite or prion and said toxic substance is a toxin or an allergen.

In yet another aspect of at least one embodiment, a method for protecting a subject against an infectious agent is provided including: a) providing autologous cells, allogenic cells or cell-like bodies; b) mixing the cells or cell-like bodies with a solution containing an expression vector encoding an antibody or an antibody fragment that binds to the infectious agent or to a toxic substance produced by the infectious agent to form a sample solution; c) loading said sample solution into at least one microfluidic device as described herein, wherein the sample is in contact with the structure; d) moving the structure within the at least one channel to pass the sample solution through the at least one constriction at least one time to transfect the cells or cell-like bodies; and e) administering to said subject with the transfected cells or cell-like bodies. In some embodiments, the method includes isolating cells from a mammal. In some embodiments, the method includes growing the cells ex vivo to increase the number of cells.

In some embodiments of the methods for protecting a subject against an infectious agent, the infectious agent is bacteria, virus, fungi, parasite or prion and said toxic substance is a toxin or an allergen.

In yet another aspect of at least one embodiment, a method for protecting a subject against an infectious agent is provided including: a) providing (or obtaining) autologous cells, allogenic cells or cell-like bodies; b) mixing the cells or cell-like bodies with a solution containing an expression vector encoding an antibody or an antibody fragment that binds to the infectious agent or to a toxic substance produced by the infectious agent to form a sample solution; c) loading said sample solution into at least one microfluidic device as described herein, wherein the sample is in contact with the structure; d) moving the structure within the at least one channel to pass the sample solution through the at least one constriction at least one time to transfect the cells or cell-like bodies; thereby preparing a cell or cell-like body for use in preventing an infection caused by an infectious agent or a toxic substance produced by the infectious agent. In some embodiments, the method includes isolating cells from a mammal. In some embodiments, the method includes growing the cells ex vivo to increase the number of cells.

In some embodiments of the methods for protecting a subject against an infectious agent, the infectious agent is bacteria, virus, fungi, parasite or prion and said toxic substance is a toxin or an allergen.

In yet another aspect of at least one embodiment, a method for protecting a subject against an infectious agent is provided including: a) providing (or obtaining) autologous cells, allogenic cells or cell-like bodies; b) mixing the cells or cell-like bodies with a solution containing an expression vector encoding an antibody or an antibody fragment that binds to the infectious agent or to a toxic substance produced by the infectious agent to form a sample solution; c) loading said sample solution into at least one microfluidic device as described herein, wherein the sample is in contact with the structure; d) moving the structure within the at least one channel to pass the sample solution through the at least one constriction at least one time to transfect the cells or cell-like bodies; wherein said subject is administered said transfected cells or cell-like bodies. In some embodiments, the method includes isolating cells from a mammal. In some embodiments, the method includes growing the cells ex vivo to increase the number of cells.

In some embodiments of the methods for protecting a subject against an infectious agent, the infectious agent is bacteria, virus, fungi, parasite or prion and said toxic substance is a toxin or an allergen.

In another aspect of at least one embodiment, a method for preparing CAR-T cells is provided including: a) optionally, isolating T cells from a mammal; b) providing autologous T cells or allogenic T cells; c) mixing the T cells with a solution containing at least genetic material encoding a chimeric antigen receptor to form a sample solution; d) loading the sample solution into at least one rigid container according to the assemblies described herein, wherein the sample is in contact with the plunger; and e) moving the plunger axially within the container to pass the sample solution through the at least one constriction at least one time to transfect the T cells.

In another aspect of at least one embodiment, a method for preparing CAR-T cells is provided including: a) optionally, isolating T cells from a mammal; b) providing autologous T cells or allogenic T cells; c) mixing the T cells with a solution containing at least genetic material encoding a chimeric antigen receptor to form a sample solution; d) loading the sample solution into at least one flexible container according to the assemblies described herein, wherein the sample is in contact with the inner surfaces of the flexible container; and e) moving at least one wedge or roller axially along the container to pass the sample solution through the at least one constriction at least one time to transfect the T cells.

In yet another aspect of at least one embodiment, a method for preparing CAR-T cells is provided including: a) optionally, isolating T cells from a mammal; b) providing autologous T cells or allogenic T cells; c) mixing the T cells with a solution containing at least genetic material encoding a chimeric antigen receptor to form a sample solution; d) loading the sample solution into at least one microfluidic device according to claim 14, wherein the sample is in contact with the structure; and e) moving the structure within the at least one channel to pass the sample solution through the at least one constriction at least one time to transfect the T cells.

In another aspect of at least one embodiment, a method for preparing CAR-T cells is provided including: a) providing (or obtaining) autologous T cells or allogenic T cells; b) mixing the T cells with a solution containing at least genetic material encoding a chimeric antigen receptor to form a sample solution; c) loading the sample solution into at least one rigid container according to the assemblies described herein, wherein the sample is in contact with the plunger; and d) moving the plunger axially within the container to pass the sample solution through the at least one constriction at least one time to transfect the T cells. In some embodiments, the method includes isolating cells from a mammal.

In some embodiments of the methods for preparing CAR-T cells, the sample solution further contains transposase enzymes, endonuclease enzymes, genetic material encoding transposase enzymes or genetic material encoding endonuclease enzymes.

In another aspect of at least one embodiment, a method for treating a subject having a disease or disorder is provided including: a) providing (or obtaining) autologous cells, allogenic cells or cell-like bodies; b) mixing the T cells with a solution containing at least genetic material encoding a chimeric antigen receptor to form a sample solution; c) loading said sample solution into at least one microfluidic device as described herein, wherein the sample is in contact with the structure; d) moving the structure within the at least one channel to pass the sample solution through the at least one constriction at least one time to transfect the cells or cell-like bodies; and e) administering said subject with the transfected cells or cell-like bodies. In some embodiments, the disease or disorder is selected from the group consisting of sickle cell anemia, severe combined immunodeficiency (ADA-SCID/X-SCID), cystic fibrosis, hemophilia, Duchenne muscular dystrophy, familial hypercholesterolemia, alpha-1 antitrypsin deficiency, chronic granulomatus disorder, Fanconi anemia, Gaucher disease, Leber's congenital amaurosis, phenylketonuria, thalassemia, oculocutaneous albinism, Huntington's disease, myotonic dystrophy, neurofibromatosis, polycystic kidney disease, hypophosphatemic rickets, Rett's syndrome, nonobstructive spermatogenic failure, fragile X syndrome, Friedreich's ataxia, spinocerebellar ataxias, Van der Woude syndrome, cancer, heart disease, diabetes, schizophrenia, Alzheimer's disease, Parkinson's disease, 22q11.2 deletion syndrome, Angelman syndrome, Canavan disease, Charcot-Marie-Tooth disease, color blindness, Cri du chat, Down syndrome, haemochromatosis, Klinefelter syndrome, Prader-Willi syndrome, spinal muscular atrophy, Tay-Sachs disease and Turner syndrome, 1p36 deletion syndrome, 18p deletion syndrome, 21-hydroxylase deficiency, 22q11.2 deletion syndrome, Alpha 1-antitrypsin deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Aicardi-Goutières syndrome, Albinism, Alexander disease, alkaptonuria, Alport syndrome, Alternating hemiplegia of childhood, Amyotrophic lateral sclerosis-Frontotemporal dementia, Alström syndrome, Alzheimer's disease, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Bjornstad syndrome, Bloom syndrome, Birt-Hogg-Dube syndrome, Brody myopathy, Brunner syndrome, CADASIL syndrome, CARASIL syndrome, Chronic granulomatous disorder, Campomelic dysplasia, Canavan disease, Carpenter Syndrome, Cerebral dysgenesis-neuropathy-ichthyosis-keratoderma syndrome (SEDNIK), Cystic fibrosis, Charcot-Marie-Tooth disease, CHARGE syndrome, Ch-diak-Higashi syndrome, Cleidocranial dysostosis, Cockayne syndrome, Coffin-Lowry syndrome, Cohen syndrome, collagenopathy (types II and XI), Color blindness, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Darier's disease, Dent's disease (Genetic hypercalciuria), Denys-Drash syndrome, De Grouchy syndrome, Down Syndrome, Di George's syndrome, Distal hereditary motor neuropathies, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Sträussler-Scheinker syndrome, Gillespie syndrome, Glutaric aciduria (type I and type 2), GRACILE syndrome, Chronic Granulomatus disorder, Griscelli syndrome, Hailey-Hailey disease, Harlequin type ichthyosis, Hereditary Haemochromatosis, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Familial Hypercholesterolemia, Hyperlysinemia, Primary Hyperoxaluria, Hyperphenylalaninemia, Hypoalphalipoproteinemia (Tangier disease), Hypochondrogenesis, Hypochondroplasia, Hypophosphatemic rickets, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Keloid disorder, Klinefelter syndrome, Kniest dysplasia, Kosaki overgrowth syndrome, Krabbe disease, Kufor-Rakeb syndrome, LCAT deficiency, Leber's congenital amaurosis, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Familial Mediterranean fever, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy (Duchenne and Becker type), Myostatin-related muscle hypertrophy, Myotonic dystrophy, Natowicz syndrome, Neurofibromatosis type I, Neurofibromatosis type II, Niemann-Pick disease, Nonketotic hyperglycinemia, Nonobstructuive spermatogenic failure, Nonsyndromic deafness, Noonan syndrome, Norman-Roberts syndrome, Oculocutaneous albinism, Ogden syndrome, Omenn syndrome, Osteogenesis imperfecta, Pantothenate kinase-associated neurodegeneration, Parkinson's disease, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thalassemia, Thanatophoric dysplasia, Treacher Collins syndrome, Tuberous sclerosis complex (TSC), Turner syndrome, Usher syndrome, Van der Woude syndrome, Variegate porphyria, von Hippel-Lindau disease, Waardenburg syndrome, Weissenbacher-Zweymüller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (a.k.a., triple X syndrome), XXXX syndrome (a.k.a., 48, XXXX), XXXXX syndrome (a.k.a., 49, XXXXX), XYY syndrome (a.k.a., 47,XYY), Zellweger syndrome, cancer, heart disease, diabetes, schizophrenia, carcinomas derived from epithelial cells (including cancers developing in the breast, prostate, lung, pancreas and colon), sarcomas arising from connective tissue (i.e. bone, cartilage, fat and nerve tissues), lymphomas and leukemia arising from cells that make blood, germ cell tumors derived from pluripotent cells and most often presenting in the testicle or ovary, and blastomas derived from immature “precursor cells or embryonic tissue”, Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer (islet cell), Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter (transitional cell cancer), Prostate cancer, Testicular cancer, Gestational trophoblastic tumor, Ureter and renal pelvis (transitional cell cancer), Urethral cancer, Uterine sarcoma, Vaginal cancer, Vulvar cancer, Wilms tumor, Esophageal cancer, Head and neck cancer, Head and neck squamous cell carcinoma, Nasopharyngeal carcinoma, Oral cancer, Oropharyngeal cancer, Paranasal sinus and nasal cavity cancer, Pharyngeal cancer, Salivary gland cancer, Hypopharyngeal cancer, Acute biphenotypic leukemia, Acute eosinophilic leukemia, Acute lymphoblastic leukemia, Acute myeloid leukemia, Acute myeloid dendritic cell leukemia, AIDS-related lymphoma, Anaplastic large cell lymphoma, Angioimmunoblastic T-cell lymphoma, B-cell prolymphocytic leukemia, Burkitt's lymphoma, Chronic lymphocytic leukemia, Chronic myelogenous leukemia, Cutaneous T-cell lymphoma, Diffuse large B-cell lymphoma, Follicular lymphoma, Hairy cell leukemia, Hepatosplenic T-cell lymphoma, Hodgkin's lymphoma, Hairy cell leukemia, Intravascular large B-cell lymphoma, Large granular lymphocytic leukemia, Lymphoplasmacytic lymphoma, Lymphomatoid granulomatosis, Mantle cell lymphoma, Marginal zone B-cell lymphoma, Mast cell leukemia, Mediastinal large B cell lymphoma, Multiple myeloma/plasma cell neoplasm, Myelodysplastic syndromes, Mucosa-associated lymphoid tissue lymphoma, Mycosis fungoides, Nodal marginal zone B cell lymphoma, Non-Hodgkin lymphoma, Precursor B lymphoblastic leukemia, Primary central nervous system lymphoma, Primary cutaneous follicular lymphoma, Primary cutaneous immunocytoma, Primary effusion lymphoma, Plasmablastic lymphoma, Sézary syndrome, Splenic marginal zone lymphoma, T-cell prolymphocytic leukemia, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor and Liposarcoma. In some embodiments, the solution further contains transposase enzymes, endonuclease enzymes, genetic material encoding transposase enzymes or genetic material encoding endonuclease enzymes.

In another aspect of at least one embodiment, a method for treating a subject having a disease or disorder is provided including: a) providing autologous cells, allogenic cells or cell-like bodies; b) mixing the T cells with a solution containing at least genetic material encoding a chimeric antigen receptor to form a sample solution; c) loading said solution into at least one microfluidic device as described herein, wherein the solution is in contact with the structure; d) moving the structure within the at least one channel to pass the solution through the at least one constriction at least one time to transfect the cells or cell-like bodies; and e) wherein said subject is administered the transfected cells or cell-like bodies. In some embodiments, the disease or disorder is selected from the group consisting of sickle cell anemia, severe combined immunodeficiency (ADA-SCID/X-SCID), cystic fibrosis, hemophilia, Duchenne muscular dystrophy, familial hypercholesterolemia, alpha-1 antitrypsin deficiency, chronic granulomatus disorder, Fanconi anemia, Gaucher disease, Leber's congenital amaurosis, phenylketonuria, thalassemia, oculocutaneous albinism, Huntington's disease, myotonic dystrophy, neurofibromatosis, polycystic kidney disease, hypophosphatemic rickets, Rett's syndrome, nonobstructive spermatogenic failure, fragile X syndrome, Friedreich's ataxia, spinocerebellar ataxias, Van der Woude syndrome, cancer, heart disease, diabetes, schizophrenia, Alzheimer's disease, Parkinson's disease, 22q11.2 deletion syndrome, Angelman syndrome, Canavan disease, Charcot-Marie-Tooth disease, color blindness, Cri du chat, Down syndrome, haemochromatosis, Klinefelter syndrome, Prader-Willi syndrome, spinal muscular atrophy, Tay-Sachs disease and Turner syndrome, 1p36 deletion syndrome, 18p deletion syndrome, 21-hydroxylase deficiency, 22q11.2 deletion syndrome, Alpha 1-antitrypsin deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Aicardi-Goutières syndrome, Albinism, Alexander disease, alkaptonuria, Alport syndrome, Alternating hemiplegia of childhood, Amyotrophic lateral sclerosis-Frontotemporal dementia, Alström syndrome, Alzheimer's disease, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Bjornstad syndrome, Bloom syndrome, Birt-Hogg-Dubé syndrome, Brody myopathy, Brunner syndrome, CADASIL syndrome, CARASIL syndrome, Chronic granulomatous disorder, Campomelic dysplasia, Canavan disease, Carpenter Syndrome, Cerebral dysgenesis-neuropathy-ichthyosis-keratoderma syndrome (SEDNIK), Cystic fibrosis, Charcot-Marie-Tooth disease, CHARGE syndrome, Chédiak-Higashi syndrome, Cleidocranial dysostosis, Cockayne syndrome, Coffin-Lowry syndrome, Cohen syndrome, collagenopathy (types II and XI), Color blindness, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Darier's disease, Dent's disease (Genetic hypercalciuria), Denys-Drash syndrome, De Grouchy syndrome, Down Syndrome, Di George's syndrome, Distal hereditary motor neuropathies, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Sträussler-Scheinker syndrome, Gillespie syndrome, Glutaric aciduria (type I and type 2), GRACILE syndrome, Chronic Granulomatus disorder, Griscelli syndrome, Hailey-Hailey disease, Harlequin type ichthyosis, Hereditary Haemochromatosis, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Familial Hypercholesterolemia, Hyperlysinemia, Primary Hyperoxaluria, Hyperphenylalaninemia, Hypoalphalipoproteinemia (Tangier disease), Hypochondrogenesis, Hypochondroplasia, Hypophosphatemic rickets, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Keloid disorder, Klinefelter syndrome, Kniest dysplasia, Kosaki overgrowth syndrome, Krabbe disease, Kufor-Rakeb syndrome, LCAT deficiency, Leber's congenital amaurosis, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Familial Mediterranean fever, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy (Duchenne and Becker type), Myostatin-related muscle hypertrophy, Myotonic dystrophy, Natowicz syndrome, Neurofibromatosis type I, Neurofibromatosis type II, Niemann-Pick disease, Nonketotic hyperglycinemia, Nonobstructuive spermatogenic failure, Nonsyndromic deafness, Noonan syndrome, Norman-Roberts syndrome, Oculocutaneous albinism, Ogden syndrome, Omenn syndrome, Osteogenesis imperfecta, Pantothenate kinase-associated neurodegeneration, Parkinson's disease, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thalassemia, Thanatophoric dysplasia, Treacher Collins syndrome, Tuberous sclerosis complex (TSC), Turner syndrome, Usher syndrome, Van der Woude syndrome, Variegate porphyria, von Hippel-Lindau disease, Waardenburg syndrome, Weissenbacher-Zweymüller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (a.k.a., triple X syndrome), XXXX syndrome (a.k.a., 48, XXXX), XXXXX syndrome (a.k.a., 49, XXXXX), XYY syndrome (a.k.a., 47,XYY), Zellweger syndrome, cancer, heart disease, diabetes, schizophrenia, carcinomas derived from epithelial cells (including cancers developing in the breast, prostate, lung, pancreas and colon), sarcomas arising from connective tissue (i.e. bone, cartilage, fat and nerve tissues), lymphomas and leukemia arising from cells that make blood, germ cell tumors derived from pluripotent cells and most often presenting in the testicle or ovary, and blastomas derived from immature “precursor cells or embryonic tissue”, Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer (islet cell), Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter (transitional cell cancer), Prostate cancer, Testicular cancer, Gestational trophoblastic tumor, Ureter and renal pelvis (transitional cell cancer), Urethral cancer, Uterine sarcoma, Vaginal cancer, Vulvar cancer, Wilms tumor, Esophageal cancer, Head and neck cancer, Head and neck squamous cell carcinoma, Nasopharyngeal carcinoma, Oral cancer, Oropharyngeal cancer, Paranasal sinus and nasal cavity cancer, Pharyngeal cancer, Salivary gland cancer, Hypopharyngeal cancer, Acute biphenotypic leukemia, Acute eosinophilic leukemia, Acute lymphoblastic leukemia, Acute myeloid leukemia, Acute myeloid dendritic cell leukemia, AIDS-related lymphoma, Anaplastic large cell lymphoma, Angioimmunoblastic T-cell lymphoma, B-cell prolymphocytic leukemia, Burkitt's lymphoma, Chronic lymphocytic leukemia, Chronic myelogenous leukemia, Cutaneous T-cell lymphoma, Diffuse large B-cell lymphoma, Follicular lymphoma, Hairy cell leukemia, Hepatosplenic T-cell lymphoma, Hodgkin's lymphoma, Hairy cell leukemia, Intravascular large B-cell lymphoma, Large granular lymphocytic leukemia, Lymphoplasmacytic lymphoma, Lymphomatoid granulomatosis, Mantle cell lymphoma, Marginal zone B-cell lymphoma, Mast cell leukemia, Mediastinal large B cell lymphoma, Multiple myeloma/plasma cell neoplasm, Myelodysplastic syndromes, Mucosa-associated lymphoid tissue lymphoma, Mycosis fungoides, Nodal marginal zone B cell lymphoma, Non-Hodgkin lymphoma, Precursor B lymphoblastic leukemia, Primary central nervous system lymphoma, Primary cutaneous follicular lymphoma, Primary cutaneous immunocytoma, Primary effusion lymphoma, Plasmablastic lymphoma, Sézary syndrome, Splenic marginal zone lymphoma, T-cell prolymphocytic leukemia, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor and Liposarcoma. In some embodiments, the solution further contains transposase enzymes, endonuclease enzymes, genetic material encoding transposase enzymes or genetic material encoding endonuclease enzymes.

In another aspect of at least one embodiment, a method for treating cancer is provided including: a) optionally, growing the T cells prepared by the methods described herein ex vivo to increase the number of cells; and b) infusing a subject in need thereof with the transfected T cells. In some embodiments of the methods for treating cancer, the cancer is a blood cancer including non-Hodgkin lymphoma or acute lymphoblastic leukemia.

In another aspect of at least one embodiment, a method for treating cancer is provided including: a) infusing a subject in need thereof with the transfected T cells. In some embodiments of the methods for treating cancer, the cancer is a blood cancer including non-Hodgkin lymphoma or acute lymphoblastic leukemia. In some embodiments, the method includes growing the T cells prepared by the methods described herein ex vivo to increase the number of cells.

In another aspect of at least one embodiment, a method for treating cancer is provided including growing the T cells prepared by the methods described herein ex vivo to increase the number of cells wherein said subject is administered the transfected cells or cell-like bodies. In some embodiments of the methods for treating cancer, the cancer is a blood cancer including non-Hodgkin lymphoma or acute lymphoblastic leukemia.

In another aspect of at least one embodiment, a method for treating a subject having a disease or condition using gene therapy is provided including: a) optionally, isolating cells from a mammal; b) providing autologous cells, allogenic cells or cell-like bodies; c) mixing the cells or cell-like bodies with a solution containing nucleic acids, proteins or mixtures thereof to form a sample solution; d) loading the sample solution into at least one rigid container according to the assemblies described herein, wherein the sample is in contact with the plunger; e) moving the plunger axially within the container to pass the sample solution through the at least one constriction at least one time to transfect the cells or cell-like bodies; f) optionally, growing the cells ex vivo to increase the number of cells; and g) infusing the subject with the transfected cells or cell-like bodies.

In another aspect of at least one embodiment, a method for treating a subject having a disease or condition using gene therapy is provided including: a) providing autologous cells, allogenic cells or cell-like bodies; b) mixing the cells or cell-like bodies with a solution containing nucleic acids, proteins or mixtures thereof to form a sample solution; c) loading the sample solution into at least one rigid container according to the assemblies described herein, wherein the sample is in contact with the plunger; d) moving the plunger axially within the container to pass the sample solution through the at least one constriction at least one time to transfect the cells or cell-like bodies; and e) infusing the subject with the transfected cells or cell-like bodies. In some embodiments, the method includes isolating cells from a mammal. In some embodiments, the method includes growing the cells ex vivo to increase the number of cells.

In another aspect of at least one embodiment, a method for treating a subject having a disease or condition using gene therapy is provided including: a) optionally, isolating cells from a mammal; b) providing autologous cells, allogenic cells or cell-like bodies; c) mixing the cells or cell-like bodies with a solution containing nucleic acids, proteins or mixtures thereof to form a sample solution; d) loading the sample solution into at least one flexible container according to the assemblies described herein, wherein the sample is in contact with the inner surfaces of the flexible container; e) moving at least one wedge or roller axially along the container to pass the sample solution through the at least one constriction at least one time to transfect the cells or cell-like bodies; f) optionally, growing the cells ex vivo to increase the number of cells; and g) infusing the subject with the transfected cells or cell-like bodies.

In another aspect of at least one embodiment, a method for preparing a cell for use in treating a subject having a disease or condition using gene therapy is provided including: a) providing autologous cells, allogenic cells or cell-like bodies; b) mixing the cells or cell-like bodies with a solution containing nucleic acids, proteins or mixtures thereof to form a sample solution; c) loading the sample solution into at least one flexible container according to the assemblies described herein, wherein the sample is in contact with the inner surfaces of the flexible container; d) moving at least one wedge or roller axially along the container to pass the sample solution through the at least one constriction at least one time to transfect the cells or cell-like bodies thereby preparing a cell for use in treating a subject having a disease or condition using gene therapy. In some embodiments, the method includes isolating cells from a mammal. In some embodiments, the method includes growing the cells ex vivo to increase the number of cells.

In yet another aspect of at least one embodiment, a method for treating a subject having a disease or condition using gene therapy is provided including: a) providing autologous cells, allogenic cells or cell-like bodies; b) mixing the cells or cell-like bodies with a solution containing nucleic acids, proteins or mixtures thereof to form a sample solution; c) loading the sample solution into at least one microfluidic device as described herein, wherein the sample is in contact with the structure; d) moving the structure within the at least one channel to pass the sample solution through the at least one constriction at least one time to transfect the cells or cell-like bodies; wherein said subject is administered the transfected cells or cell-like bodies. In some embodiments, the method includes isolating cells from a mammal. In some embodiments, the method includes growing the cells ex vivo to increase the number of cells.

In some embodiments of the methods for treating a subject having a disease or condition using gene therapy, the disease or condition is a monogenic disorder, a polygenic disorder, a neurological disease, a cardiovascular disease, an autoimmune disease, an inflammatory disease, a cancer disease, an ocular disease or an infectious disease. In some embodiments, the gene therapy includes replacing a defective or mal-adaptive gene, altering or killing an aberrant cell, or inducing production of a therapeutic protein.

In some embodiments, the disease or condition is a monogenic disorder or a polygenic disorder including: sickle cell anemia, severe combined immunodeficiency (ADA-SCID/X-SCID), cystic fibrosis, hemophilia, Duchenne muscular dystrophy, familial hypercholesterolemia, alpha-1 antitrypsin deficiency, chronic granulomatus disorder, Fanconi anemia, Gaucher disease, Leber's congenital amaurosis, phenylketonuria, thalassemia, oculocutaneous albinism, Huntington's disease, myotonic dystrophy, neurofibromatosis, polycystic kidney disease, hypophosphatemic rickets, Rett's syndrome, nonobstructive spermatogenic failure, fragile X syndrome, Friedreich's ataxia, spinocerebellar ataxias, Van der Woude syndrome, cancer, heart disease, diabetes, schizophrenia, Alzheimer's disease, Parkinson's disease, epilepsy, 22q11.2 deletion syndrome, Angelman syndrome, Canavan disease, Charcot-Marie-Tooth disease, color blindness, Cri du chat, Down syndrome, haemochromatosis, Klinefelter syndrome, Prader-Willi syndrome, spinal muscular atrophy, Tay-Sachs disease or Turner syndrome.

In some embodiments, the infectious disease results from a chronic viral, mycobacterial, bacterial or parasitic infection. In some embodiments, the infectious disease is HIV/AIDS, hepatitis, malaria, herpes, Burkholderia, Creutzfeldt-Jacob or human papillomavirus.

In some embodiments, the cancer disease is head and neck cancer, prostate cancer, pancreas cancer, brain cancer, skin cancer, liver cancer, colon cancer, breast cancer, kidney cancer or mesothelioma.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In addition, the materials, methods, and examples are illustrative only and not intended to be limiting.

Where applicable or not specifically disclaimed, any one of the embodiments described herein are contemplated to be able to combine with any other one or more embodiments, even though the embodiments are described under different aspects of the disclosure.

These and other embodiments are disclosed and/or encompassed by, the following detailed description.

### BRIEF DESCRIPTION OF THE DRAWINGS

FIG. 1 is a schematic diagram of a transfection system.

FIGS. 2A-E illustrate various container/plunger assembly embodiments.

FIGS. 2F-J illustrate various views (side and top) of a planar container/plunger assembly.

FIG. 2K illustrates a planar container/plunger assembly where the plungers are flexible sheets coupled to piezoelectric stacks.

FIG. 2L illustrates an alternative container assembly embodiment.

FIGS. 2M-O illustrate mass fabrication schemes for high density container/plunger systems on planar structures.

FIGS. 2P and Q illustrate container assembly embodiments having multiple constrictions.

FIG. 2R illustrates a container assembly embodiment having an insert with multiple constrictions.

FIGS. 2S-U illustrate alternative container assembly embodiments.

FIGS. 3A and B illustrate plungers.

FIG. 3C illustrates a plunger inserted into a container.

FIG. 3D illustrates an alternative container assembly embodiment with plunger inserted.

FIG. 4 illustrates a housing for a transfection system including a container/plunger assembly.

FIG. 5 illustrates plunger position settings during a transfection process.

FIGS. 6A-C illustrate alternative plunger/container assembly embodiments.

FIGS. 7A-C illustrate various views of a heating unit.

FIG. 8 is a schematic diagram of a container constriction formation system.

FIG. 9 shows photographs of NIH/3T3 cells (left panel: light microscopy; right panel: fluorescent microscopy) showing expression of GFP 4 weeks after transfection with a 4.7 kb plasmid expression vector. NIH/3T3 cells were transfected with 15 μg pAcGFP vector (4.7 kb) in complete medium using a 50RL capillary; 15 cycles at a flow rate of 47/47 microliters per second. Transfection efficiency was about 10%.

FIG. 10 shows photographs of NIH/3T3 cells (left panels: light microscopy; right panels: fluorescent microscopy) showing expression of nuclear localized green fluorescence 6 hours and 24 hours post-transfection with an Alexa Fluor 488 labeled 22 kDa protein. NIH/3T3 cells were transfected with 22 kDa protein conjugated to Alexa Fluor 488. Transfection was performed using a 50RL capillary with 100,000 cells in 100 μl transfection solution with 8 μg protein for 15 cycles at a flow rate of 30/30 microliters per second. Transfection efficiency was greater than 95%.

FIG. 11 shows photographs of HeLa cells (left panels: light microscopy; right panels: fluorescent microscopy) showing expression of nuclear localized green fluorescence 6 hours and 24 hours post-transfection with an Alexa Fluor 488 labeled 22 kDa protein. Transfection was performed using a 50RL capillary with 100,000 cells in 100 μl transfection solution with 8 μg protein for 15 cycles at a flow rate of 30/30 microliters per second. Transfection efficiency was greater than 95%.

FIG. 12 shows photographs showing the effect of flow rates on cell survivability. Approximately 100,000 NIH/3T3 cells were suspended in DMEM complete medium containing 10% Fetal Bovine serum and passed through a 50RL capillary for 15 cycles at a flow rate of 45/45, 70/70 and 100/100 microliters per second. The flow rate values indicate inward and outward flow rates. Cells were imaged within 2 hours and 24 hours after transfection. As the flow rate increased the cell count was lowered. The 24-hour time point indicates that the cells were able to survive the procedure and undergo regular proliferation.

FIG. 13 shows photographs showing the effect of flow rates on cell survivability. Approximately 100,000 NIH/3T3 cells were suspended in Dulbecco's Phosphate Buffered Saline (DPBS) and passed through a 50RL capillary for 15 cycles at a flow rate of 45/45, 70/70 and 100/100 microliters per second. The flow rate values indicate inward and outward flow rates. Cells were imaged within 2 hours and 24 hours after transfection. As the flow rate increased the cell count was lowered. The 24-hour time point indicates that the cells were able to survive the procedure and undergo regular proliferation.

FIG. 14 shows photographs showing the effect of flow rates on cell survivability. Approximately 100,000 NIH/3T3 cells were suspended in DMEM complete medium containing 10% Fetal Bovine serum and passed through a 80RL capillary for 15 cycles at a flow rate of 70/70, 100/100 and 114/114 microliters per second. The flow rate values indicate inward and outward flow rates. Cells were imaged within 2 hours and 24 hours after transfection. As the flow rate increased the cell count was lowered. The 24-hour time point indicates that the cells were able to survive the procedure and undergo regular proliferation.

FIG. 15 shows photographs showing the effect of flow rates on cell survivability. Approximately 100,000 NIH/3T3 cells were suspended in Dulbecco's Phosphate Buffered Saline (DPBS) and passed through a 80RL capillary for 15 cycles at a flow rate of 70/70, 100/100 and 114/114 microliters per second. The flow rate values indicate inward and outward flow rates. Cells were imaged within 2 hours and 24 hours after transfection. As the flow rate increased the cell count was lowered. The 24-hour time point indicates that the cells were able to survive the procedure and undergo regular proliferation.

FIG. 16 shows photographs of unmanipulated control cells. Approximately 100,000 NIH/3T3 cells were suspended in DMEM complete medium containing 10% Fetal Bovine serum or Dulbecco's Phosphate Buffered Saline (DPBS), but not passed through a capillary. Cells were imaged within 2 hours and 24 hours after plating.

FIG. 17 shows a diagram of portions of a mammalian expression vector containing the human elongation factor 1 (EF1a) promoter functionally linked to cDNA encoding a variable heavy chain (VH) and a variable light chain (VL) that bind botulinum neurotoxin serotype A (BoNT/A), the VH and VL separated by a linker sequence, and a bovine growth hormone (BGH) poly-adenylation sequence.

FIG. 18 is a diagram of portions of a mammalian expression vector containing a functional cassette encoding anti-CD19 CAR including the EF-1a promoter, anti-CD19 scFV cDNA, a spacer sequence, human CD8a transmembrane domain, CD28 intracellular signaling domain, the gamma chain of Fc epsilon RI and a BGH poly-adenylation sequence; and a second functional cassette encoding enhanced green fluorescent protein (EGFP) including the cytomegalovirus promoter functionally linked to cDNA encoding EGFP and a BGH poly-adenylation sequence.

FIGS. 19A and B are (A) a diagrammatic representation of the germline map of SERPINA1 gene loci (https://www.ncbi.nlm.nih.gov/gene/5265) and (B) a diagrammatic representation of a DNA construct with a cMyc tag sequence functionally linked to SERPINA1 gene.

FIG. 20 is a schematic diagram of a process flow example with multiple sensors.

FIG. 21 is a schematic diagram of a process flow example with one sensor and one feedback control loop.

FIG. 22 is a series of photographs of human T cells (left panels: phase images; right panels: fluorescent microscopy images) showing expression of GFP after transfection with 4.7 kb pAcGFP vector.

FIG. 23 illustrates a system including a container (e.g. capillary) and an impeller pump.

FIG. 24 is a bar graph showing the results of a CAR-T cell kill assay.

### DETAILED DESCRIPTION

The present disclosure is based, at least in part, on a method of transferring molecules in a solution into cells or cell-like bodies by passing the molecules and cells or cell-like bodies through a constriction. The present disclosure provides devices, systems and methods for performing transfections. Successful transfection occurs when the appropriate constriction diameter or cross-sectional area (larger than the cells so that the cells are not mechanically squeezed) is combined with: (a) a plunger in combination with a container, where the plunger is in contact with the sample solution, and/or (b) the specific way in which the constriction is formed (i.e. its geometry), e.g. short or long distances between the minimum and maximum diameters or cross-sectional areas of the container, and/or (c) how the sample solution is pulled into the container and pushed back out again (e.g., flow rates and holding times), and/or (d) the roughness of the surface of the inner walls of the container, and/or (e) the geometry of the cross-sectional area of the constriction (e.g. circular or polygonal). The devices, systems, kits and methods provided herein are important because they provide high transfection efficiency, high cell viability, low variability, low cell toxicity, fast cell recovery and the ability to transform a multitude of cell sizes and types.

### Device/Instrument/System

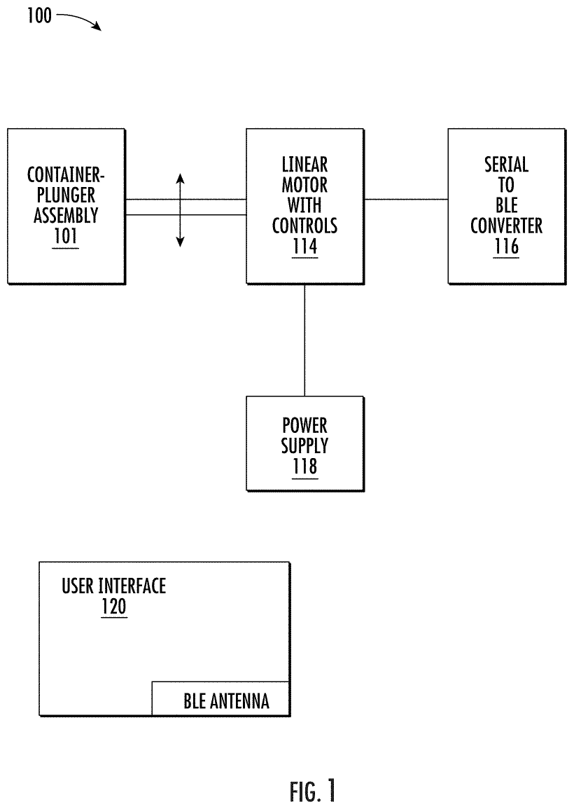

A transfection system 100, shown schematically in FIG. 1, is disclosed. The system 100 generally includes a container/plunger assembly 101 comprising a transfection chamber or container (for example, a capillary tube) and a plunger. The assembly 101 is connected to a motor 114 (for example, a linear motor) such that the motor causes the plunger to move back and forth within the container in a linear motion, as described in more detail below. The motor 114 is electrically connected to a power supply 118 and is controlled by a user-programmable unit 116 connected to a user interface 120. In some embodiments, the user interface 120 may be a mobile personal computer, tablet or smart phone. The user interface 120 may communicate with the programmable unit 116 via a network connection. In some embodiments, the network connection could be a short-distance, wireless technology, such as Bluetooth®. However, other types of user interfaces 120 and network connections are contemplated by this disclosure. The system 100 is configured to be at least partially enclosed within a housing, as further described below.

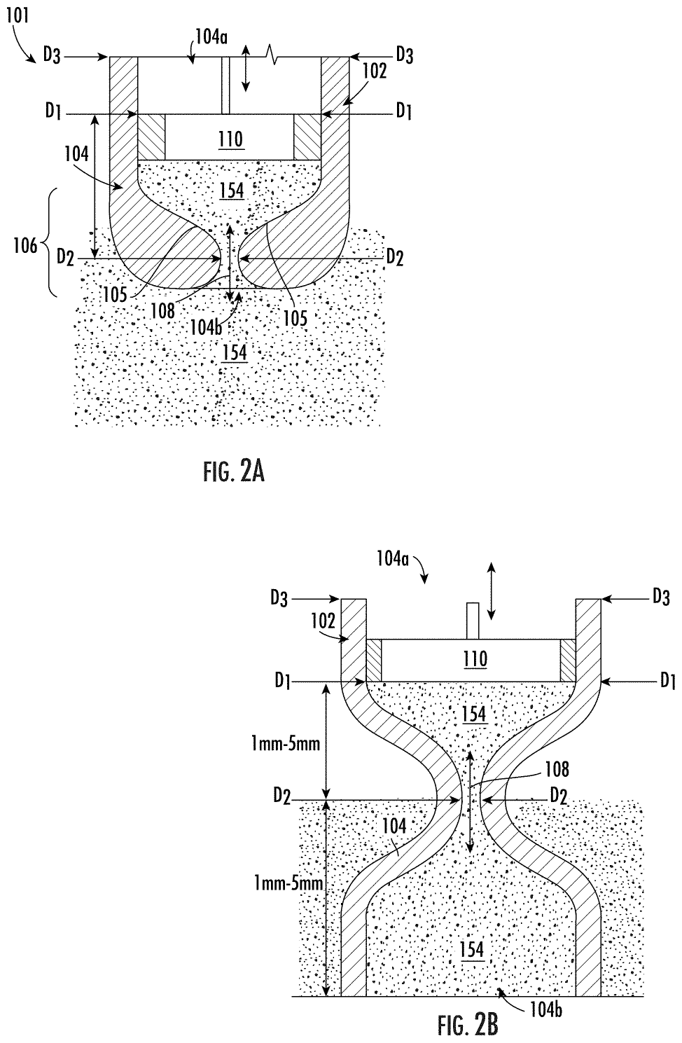

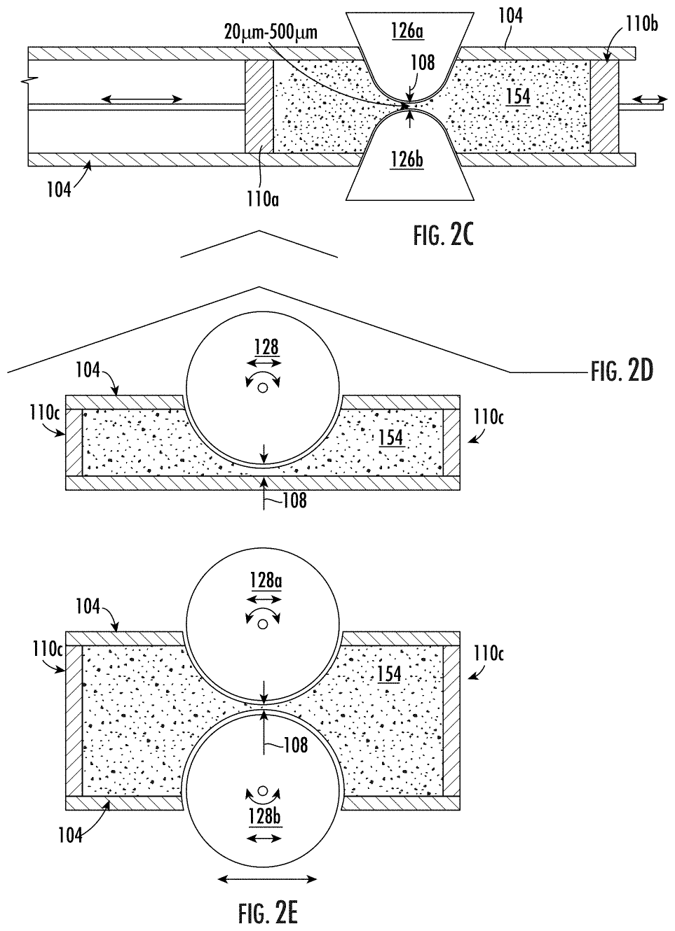

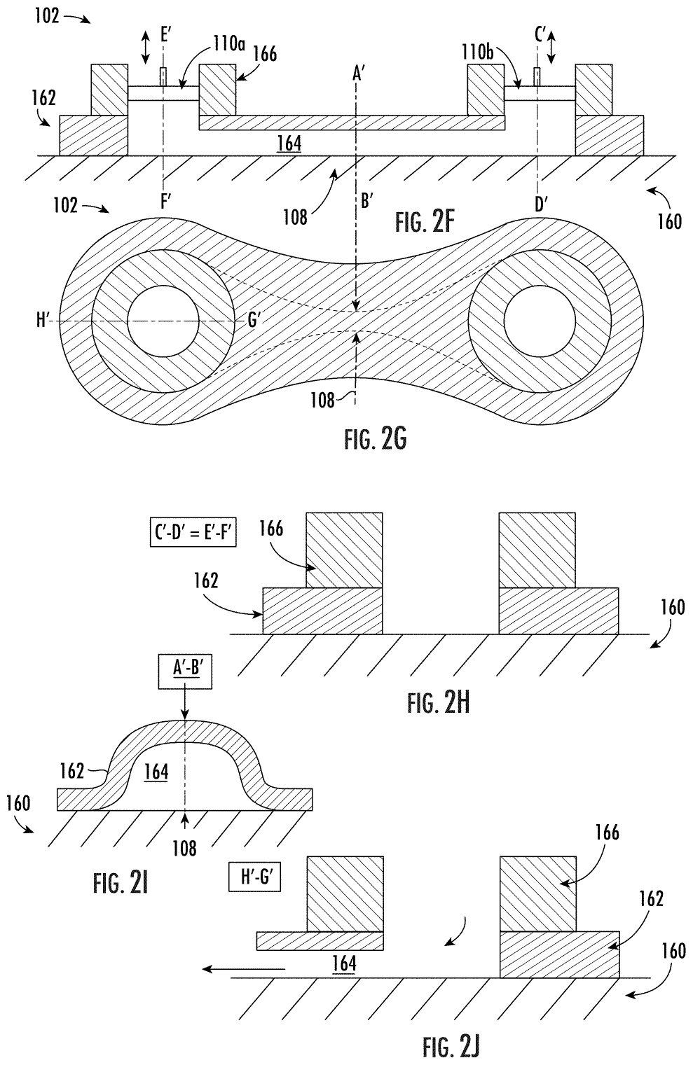

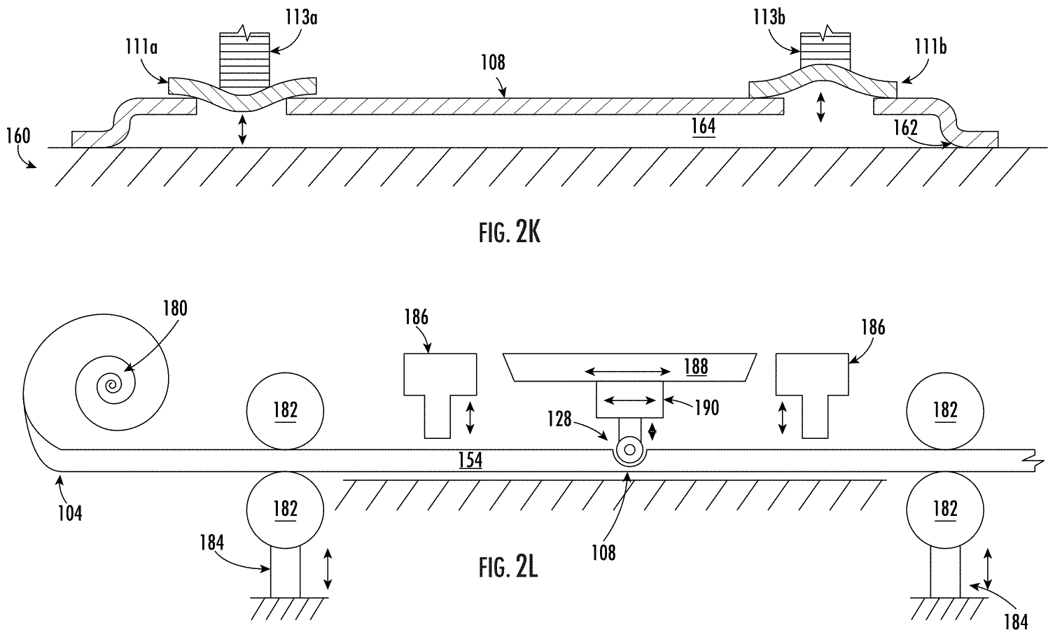

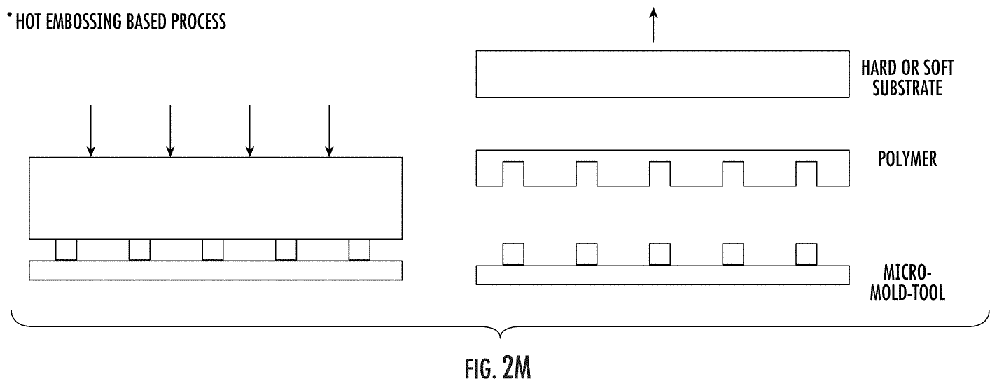

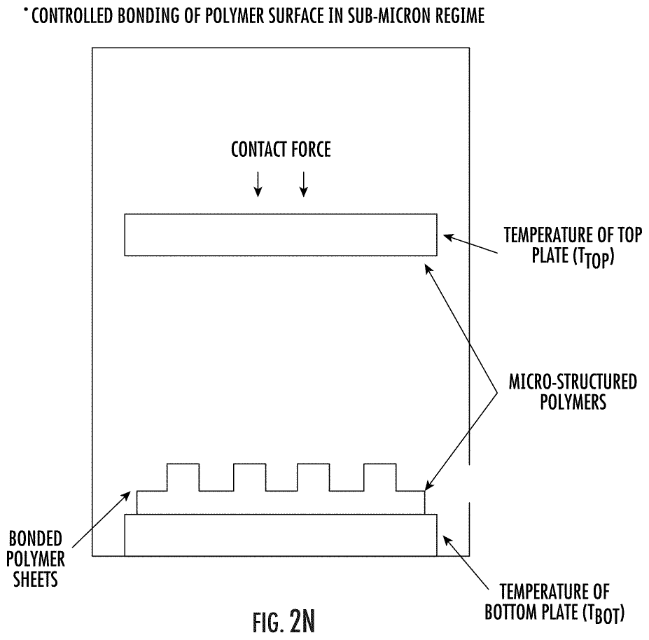

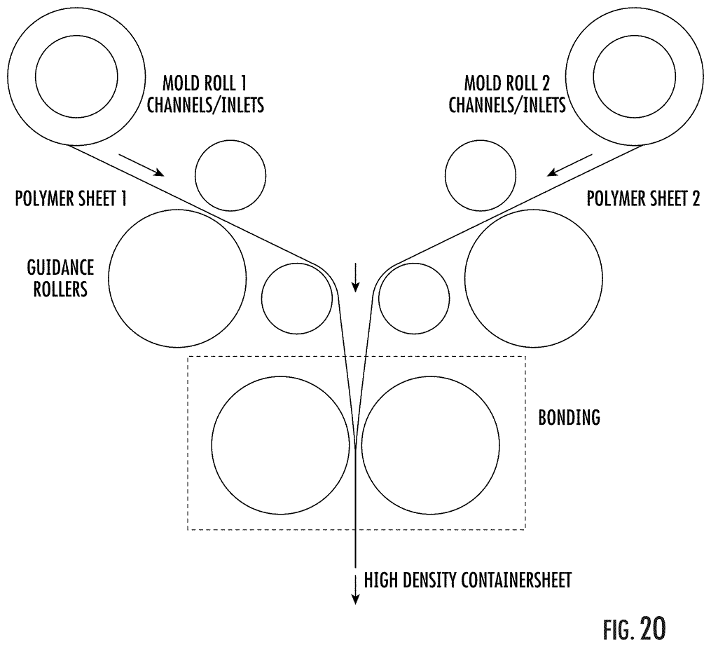

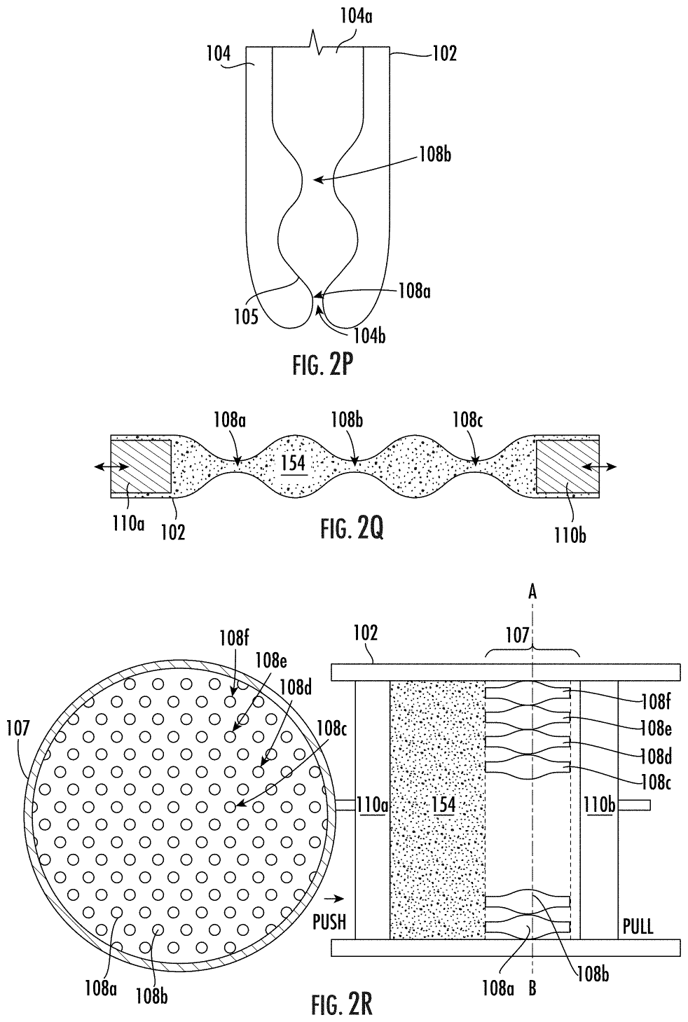

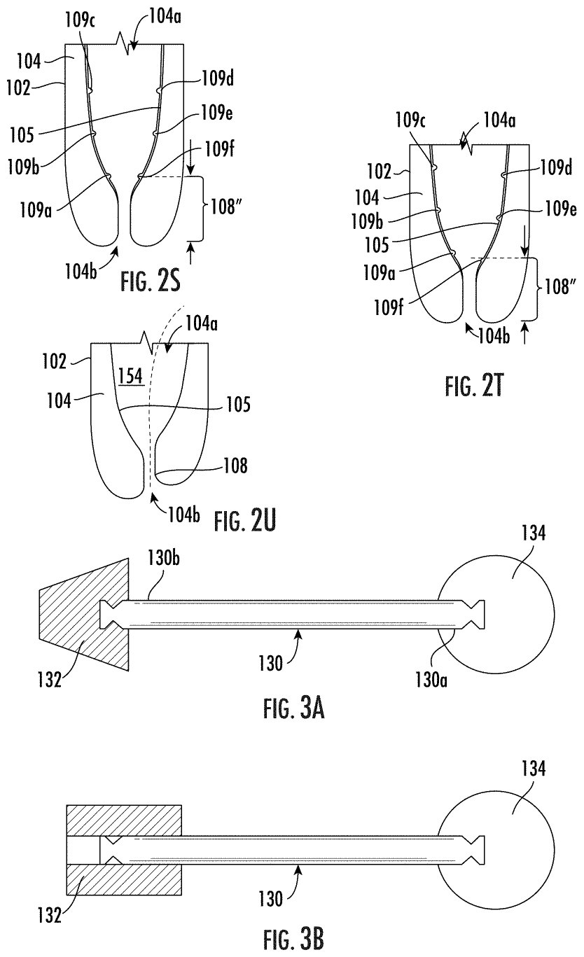

Turning now to FIG. 2A, a first, non-limiting embodiment of a container/plunger assembly 101 for use in the transfection system 100 is shown. The container/plunger assembly 101 generally comprises a container 102 and a plunger 110 insertable within the container 102. In some embodiments, the container 102 includes a hollow, cylindrically-shaped body 104 made of a rigid material (such as borosilicate glass) having an open proximal end 104a and an open distal end 104b. However, other shapes of the body 104, such as polygonal shapes, are contemplated by this disclosure. The proximal end 104a of the body 104 has a first diameter Di defined by the inner walls 105 of the body 104. In some embodiments, the first diameter Di is about 5.0 μm to about 100.0 mm, preferably about 2.2 mm. The distal end 104b of the body includes a tip 106 for insertion into a sample solution 154. As shown in FIG. 2A, at least one of the opposing inner walls 105 of the tip 106 narrows toward the distal end 104b of the body 104 (for example, over a distance of 0.2 mm to 10 mm) such that the tip 106 defines a constriction 108 having a second diameter D₂ selected to be smaller than the first diameter Di. Towards the end of the tip 106 at the distal end 104b, the constriction 108 then widens. In the embodiment of FIG. 2A, both of the inner walls 105 narrow to an equal thickness such that the constriction 108 is disposed along a central axis of the body 104. However, it is contemplated that only one of the inner walls 105 could narrow such that the constriction 108 is offset from the central axis of the body 104. Notably, the minimum diameter D₂ of the constriction 108 is selected to be 1.2 to 100 times larger than the diameter of the cells being transfected. That is, cell diameters typically range from about 4.5 μm (rat whole blood cells) to about 120 μm (human oocytes). Therefore, the minimum diameter D₂ of the constriction 108 is selected to range from about 5.4 μm to about 12000 μm (i.e., about 0.0054 mm to about 12.0 mm).

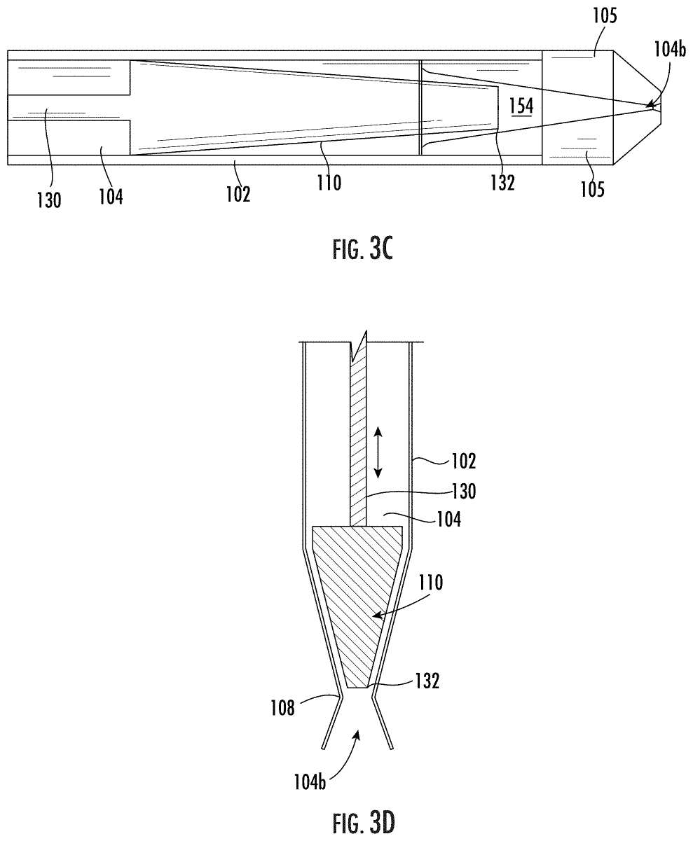

In some embodiments, the flow path length on each side of the minimum diameter of the constriction 108 is about 0.2 mm to 10 mm. The flow path distance at the minimum diameter constriction 108 can be 0.1 μm to 10 mm. The plunger 110 is configured to be insertable through the proximal end 104a of the container 102 and axially movable within the container 102. Embodiments of the plunger 110 will be described in more detail with regard to FIGS. 2 and 3.

In some embodiments, the inner walls of the container including the constriction section are roughened to control gas sphere density and size during the transfection process. The surface roughness controls and changes the boundary condition of the flow and therefore the stress/energy applied to the cells. Depending on the roughness, the local flow at the interface between the sample solution and the container can be changed from laminar to non-laminar, which impacts the constriction size and flow rate requirements needed to achieve optimum transfection results. The average roughness number of the inner wall surface of the container can range from 1 nm to 10 μm, and more specifically from 10 nm to 1 μm. The inner walls of the container can be roughened by known mechanical or chemical roughening methods such as etching, sand blasting, molding, adsorption of molecules or particles to the surface or chemical linkage of molecules or particles to the surface.

In one embodiment, the surface roughness was created by adsorbing molecules onto the surface, which increased the transfection rate significantly. In certain embodiments, the surface roughness was created by adsorbing cell fragments to the inner wall of the container near the constriction. Transfection efficiency was increased significantly (more than 50% improvement) as assessed qualitatively with optical microscopy. The roughness was at similar dimensions as the cell diameters, i.e. in the range of 1-10 μm.

FIGS. 2B-E illustrate alternative methods of forming the constriction 108 in the container/plunger assembly 101. In FIG. 2B, the constriction 108 is formed along the central axis of the body 104 by a narrowing of the outer diameter D₃ of the body 104 such that the outer diameter D₃ of the body 104 forms an “hourglass” shape. For example, the outer diameter D₃ may narrow over a distance of 1 mm to 5 mm, and then widen again over a distance of 1 mm to 5 mm to the distal end 104b of the body 104. In FIG. 2C, the body 104 comprises a flexible material such as metals, nitrides, oxides, carbides and polymers. Representative examples of polymers that can be used for the body include polypropylene, polyethylene, polyurethanes, polycaprolactone, latex and other elastomers. At least one wedge 126 is clamped from one side or from opposite sides into the body 104 to form the constriction 108. One plunger 110a is positioned close to the constriction 108 and another plunger 110b is positioned on the opposite side of the constriction 108. A distance between the plungers 110a,b can vary based on the desired volume of the sample solution 154. Both plungers 110a,b are in contact with the sample solution 154, and both plungers 110a,b are moved in the same direction to drive the sample solution 154 through the constriction 108. Once the sample solution 154 has passed through the constriction 108, the plungers 110a,b are each moved in the opposite direction. This back and forth movement can be repeated for the desired number of cycles. In other embodiments, the plungers are replaced with caps 110c fixed in position as the wedges 126a,b forming the constriction 108 move along the body 104, as long as there is relative linear motion between the caps 110c and the constriction 108. The shape, size and position of the wedges 126a, b can be adjusted to change the size of the constriction.

In FIG. 2D, the body 104 is made of a flexible material and both the proximal end 104a and the distal end 104b are sealed with a plug or cap after being filled with the sample solution 154. The constriction 108 is formed by a roller 128 positioned along the body 104, and which is lowered onto the body 104, creating a moving constriction 108 offset from a central axis of the body 104 between two fixed caps 110c. In this embodiment, the roller 128 is moved laterally along the length of the body 104, which forces the sample solution 154 to flow through the constriction 108. Alternatively, as shown in FIG. 2E, two rollers 128a,b can be used to roll in tandem along a length of the body 104, creating a moving constriction 108 along the central axis of the body 104 between two fixed caps 110c. Thus, in these embodiments, the function of the plunger is performed by the rollers 128, 128a, 128b, which when moved, force the solution in the container 104 through the constriction 108.

In other embodiments, FIGS. 2F-2K, the container 102 is designed on a planar surface or substrate 160 using miniaturization and microfabrication techniques known to those skilled in the art, such as SU8 structures, surface micromachining with other additive layers, e.g. SiO₂, Si₃N₄, graded surface etching for non-rectangular structures (ion milling), Si-Bulk micromachining, by DRIE, Si-embedded cavity technologies (BOSCH), or micro mold and micro printing techniques. Such containers 102 (e.g. microfluidic devices) are configured to be used for transfections involving very small sample solution volumes (e.g. 10 μl or less). The container 102 includes one or more flow paths or channels 164 with one or more constrictions 108 formed by planar structures 162. In all embodiments, the plungers 110a,b are configured to be insertable into the container 102 to move the sample solution 154 through the one or more constrictions 108. The container includes a tube connection 166, which forms an interface between the microfluidic structure of the device and a pre-transfection sample solution reservoir. There is also a tube connection interface located at the other end of the microfluidic structure which contains a constriction; this tube connection forms an interface between the microfluidic structure and a post-transfection sample collection reservoir. FIG. 2F is the cross-sectional view of the design of the transfection device using thick and thin film fabrication processes; FIG. 2G is the top view, and FIGS. 2I-J are the cross sections of specific areas/parts of FIG. 2F. In some embodiments, FIG. 2K, the plungers are flexible sheets 111a, b that are in contact with and powered by piezoelectric stacks 113a,b. In some embodiments, a piezo-electric interaction is provided by a surface acoustic wave device. The flexible sheets can be made of inorganic materials such as nitrides, oxides, metals and polymers. Representative polymers that can be used include polypropylene, polyethylene, polyurethanes and polycaprolactone. It is additionally contemplated that post-transfection, the sample will be collected in a bulk reservoir which is connected on the other end of the constriction part of the microfluidic devices described herein. In yet other embodiments of FIGS. 2F-2K, the container 102 is designed as a flow-through system in order to perform transfections using large sample volumes (e.g. several liters). In large sample volume systems, the tubes 166 would be extended or would lead into large containers.

In FIG. 2L, the body 104 is made of a flexible material filled with the sample solution 154. The constriction 108 is formed by a roller 128 positioned along the body 104, and which is lowered onto the body 104, creating a moving constriction 108 offset from a central axis of the body 104. In this embodiment, the roller 128 is moved laterally along the length of the body 104 by moving a shuttle assembly 188/190, which forces the sample solution 154 to flow through the constriction 108. In addition or alternatively, the body 104 is moved along a lateral plane by drive rollers 182, which forces the sample solution 154 to flow through the constriction 108. The motion of the sample solution 154 within the body 104 can be stopped by depressing external shutoff mechanisms 186 to fully close the body 104. The drive rollers 182 can be retracted by depression/retraction mechanisms 184 in order to move the body 104 out of the drive rollers 182 to collect a post-transfection sample. The body 104 is filled with sample solution 154 through an opening 180 at the proximal or distal (not shown) end. The functional operation of the device as shown in FIG. 2L is equivalent to that of the device as shown in FIG. 2D.

FIGS. 2M-O illustrate fabrication schemes that enable mass fabrication of container/plunger systems in a planar fashion serial sheet fabrications up to roll to roll fabrication. FIG. 2M shows an embossing process which could be supported with heat or a polymer cross linkage step to transfer the channel pattern from the tool in a sheet shaped thermoplastic or cross linkable polymer sheet. Massive parallel channel systems can be manufactured.

FIG. 2N shows a bonding process step based on methods such as thermal bonding, adhesive bonding, or solvent bonding. One or both sheets can be pre-formed. Additionally, more than one layer can be bonded to form a 3D channel system in a planar fashion. By using two pre-formed sheets having channels with cross sections of a half circle, circular cross-section container structures can be made if desired.

FIG. 2O shows how the fabrication scheme can be translated to a roll to roll process.

The plungers can be inserted before or after the bonding process step directly within the channels or connected through formed inlets of the polymer sheets. Thermoplastic cross linkable polymers can be used, preferably materials which are biocompatible are used.

FIG. 2P illustrates a container 102 having more than one constriction 108a and 108b. The plunger (not shown) is inserted at the proximal end 104a. This is an illustrative example with two constrictions, but containers with more than two constrictions are also envisioned. The multiple constrictions can be the same diameter or cross-sectional area or different diameters or cross-sectional areas. One advantage of having different diameters or cross-sectional areas is that cells of different sizes can be transfected simultaneously so long as the smallest constriction is large enough to avoid any mechanical squeezing or constraints of the largest cells within the sample.

FIG. 2Q illustrates a container 102 having multiple constrictions 108a, 108b, 108c and a plunger at each end 110a and 110b. The plungers are moved in the same direction to pass the sample solution 154 containing cells and molecules to be transfected through the multiple constrictions. This is an illustrative example with three constrictions, but containers with varying numbers of constrictions are also envisioned. The multiple constrictions can be the same diameter or cross-sectional area or different diameters or cross-sectional areas.

FIG. 2R shows a side view of a container 102 having multiple constrictions 108a-f and two plungers 110a and 110b that move in the same direction to pass the sample solution 154 containing cells and molecules to be transfected through the multiple constrictions. The constrictions are disposed within a removable insert 107. A cross-sectional view (from A to B) of the insert 107 shows multiple constrictions 108a-f and others unlabeled. The insert includes multiple containers or multiple channels, each container or channel having at least one constrictions. The multiple constrictions can be the same diameter or cross-sectional area or different diameters or cross-sectional areas. This illustrative embodiment is advantageous for transfecting large sample volumes.

FIGS. 2S and 2T illustrate containers 102 having interior walls 105 that are not smooth, but rather include ripples 109a-f that protrude away from the interior wall 105 into the interior space of the container 102 where the sample solution 154 can be contained. In FIG. 2S, the ripples 109a-f are shown in a “circular” configuration, i.e. in the cross-sectional view as shown the ripples 109a-f are not directly opposite one another so that in 3D space the ripples 109a-f form a series of circles in a cylindrical container 102. In FIG. 2T, the ripples 109a-f are shown in a “spiral” configuration, i.e. in the cross-sectional view as shown the ripples 109a-f do not appear to be directly opposite one another so that in 3D space the ripples 109a-f form a spiral in a tubular container 102. Concerning the minimal constriction 108, FIG. 2S shows a constriction 108″ over a relatively long linear distance (1 mm or more) as compared to FIG. 2T showing a constriction 108′ over a shorter linear distance (less than 1 mm). It is envisioned that these features can be varied and interchanged, e.g. a container may have spiral ripples paired with a long linear distance constriction or a container may have circular ripples paired with a short linear distance constriction.

FIG. 2U illustrates a container 102 having smooth interior walls 105, a minimum constriction located at the outlet, i.e. the distal end 104b of the container 102. In this representative example, the inner walls 105 of the container are asymmetrical.

In the illustrated containers 102 of FIGS. 2S-2U, the plunger 110 (not shown) is insertable at the proximal end 104a with a starting position as close as possible to the distal end 104b. In some embodiments, a sample solution 154 is pre-loaded into such containers 102 so that it is in contact with the plunger 110 in order to obtain high transfection results i.e., in order to obtain a high number of transfected cells or cell-like bodies, for example more than 30% of the cells.

Turning now to FIGS. 3A and 3B, embodiments of the plunger 110 of the container/plunger assembly 101 are shown in a side view. As shown in FIG. 3A, the plunger 110 comprises a rod 130 having a proximal end 130a and a distal end 130b. The rod 130 may be comprised of a rigid material, such as stainless steel or plastic. A tip 132 is coupled to the distal end 130b of the plunger 110. The tip 132 may be comprised of a polymer having “non-stick” properties, such as polyimides or Teflon™, as well as biocompatible polymers such as polypropylene, polyethylene, polyurethanes and polycaprolactone and biodegradable materials such as poly(lactide-co-glycolide) (PLGA), polylactide (PLA), poly(glycolic acid) (PGA), polyethylene glycol (PEG) and collagen. The tip 132 may have a conical shape (FIG. 3A) or a cylindrical shape (FIG. 3B) and is dimensioned to be inserted into the container 102 as close as possible to the constriction 108 and to form an air and liquid tight seal within the first diameter D¹ of the container 102. A length of the tip 132 may be between about 1 mm and 3 mm. The proximal end 130a of the rod 130 has an attachment 134 configured to attach the plunger 110 to a motorized arm 140 (not shown) of the transfection system 100, as further described below. An overall length of the plunger 110 is selected such that the proximal end 130a of the plunger 110 extends beyond the open proximal end 104a of the container 102 when the plunger 110 is fully inserted into the container 102 such that the proximal end 104a of the container 102 does not limit the movement of the motorized arm 140. When inserted into the container 102, the tip 132 of the plunger 110 is in contact with the sample solution 154 such that there is no air present at the sample solution-plunger interface. Likewise, in embodiments where the plungers are flexible sheets 111a,b powered by piezoelectric stacks 113a,b, the flexibles sheets 111a,b are in contact with the sample solution 154 such that there is no air present at the sample solution-flexible sheet interface.

In the embodiments described above, FIGS. 2A-L, the containers or channels are cylindrical in shape, and the term diameter is used in its ordinary sense. That is, the diameter of a cross section of a cylindrical container or diameter refers to a line segment which passes through the center of a circle, and whose end points lie on the circle. In alternative embodiments, the containers or channels can be elliptical or polygonal in shape. Representative examples of polygonal shapes include triangular, square, rectangular, pentagonal, hexagonal, heptagonal, octagonal, etc. In the case of polygonal containers or channels, the cross-sectional area is 1.5 to 10,000 times larger than the cross-sectional area of the cells being transfected. In the case of polygonal containers or channels having an even number of sides, the minimum distance between opposite walls is at least 1.2 times larger than the cell diameter. In all cases, the minimum size of the container or channel through which the cells pass must be large enough to avoid any mechanical squeezing or constraints of the cells as they pass through the constriction.

FIGS. 3C and 3D show embodiments of the container/plunger assembly 101 with a plunger 110 inserted into a container 102. In some embodiments, the container 102 is molded using thermoplastic materials. The tip 132 of the plunger 110 is conical in shape with a blunt end (FIG. 3C) or a pointed end (FIG. 3D). The plunger 110 including the tip 132 is molded using thermoplastic materials. In FIG. 3D, the plunger/tip 110/132 is molded to fit the constriction section of the container 102 such that there is little or no space between the plunger/tip 110/132 and the inner walls of the container.

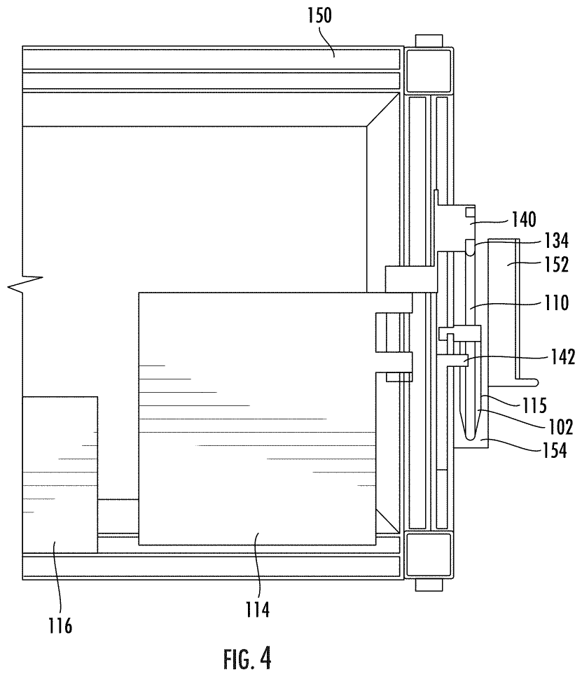

Turning now to FIG. 4, an embodiment of a housing 150 for containing and operating the container/plunger assembly 101 is shown in a transparent view. The housing 150 may be sized to be located primarily on a bench or table, or alternatively is mobile. As shown in FIG. 4, the housing 150 houses the motor 114 and the user-programmable unit 116 of the transfection system 100. The motor 114 is coupled to a movable arm 140 such that the motor 114 moves the arm 140 in a linear “back-and-forth” motion. The arm 140, in turn, is coupled to the attachment 134 of the plunger 110, such that the arm 140 moves the plunger 110 in a linear motion through the container 102. The container 102 may be secured against the housing 150 by a clamp 142 or other structure such that the tip 106 of the container 102 is in contact with the sample solution 154. A shield 152 may be attached to the housing 150 for protection of the container 102. In some embodiments, the transfection system 100 includes a sample reservoir 115 for holding the sample solution 154 before and after passage through the container/plunger assembly 101.

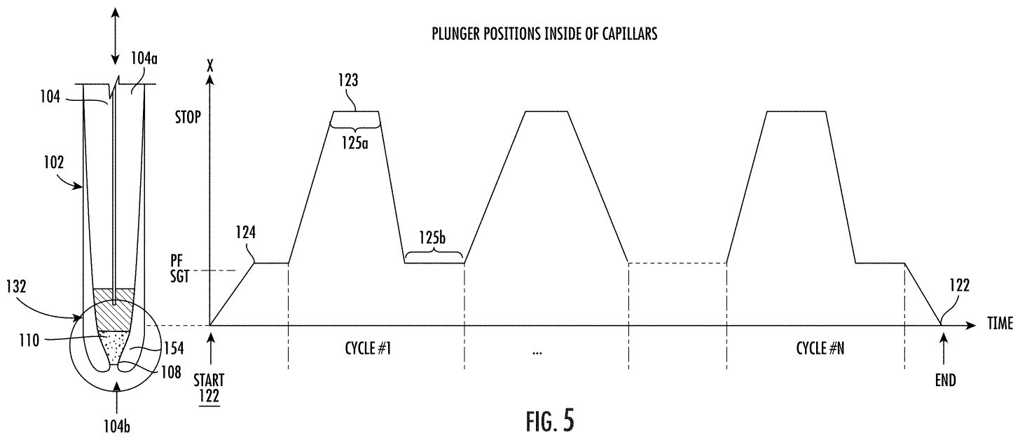

Turning now to FIG. 5 in embodiments, the user interface 120 is used to set the starting position 122 of the plunger 110 within the body 104 of the container 102. In the starting position 122, the plunger 110 is inserted at the proximal end 104a of the body 104 and is advanced to an area as close to the constriction 108 as possible (i.e., to the point where the inner wall 105 of the container 102 begins to constrict). The volume between the tip 132 of the plunger 110 and an end of the constriction 108 is pre-filled with sample solution 154 so that there is no air space between the sample solution 154 and the plunger 110. Alternatively, the volume between the tip 132 of the plunger 110 and an end of the constriction 108 is pre-filled with sample solution 154 minus the cells to be transfected (i.e., a buffer or media solution). In some embodiments, such pre-filling is preferred as it is important to the transfection outcome that the plunger(s) be in direct contact with the sample solution. If there is an airgap between the plunger and the sample solution then the flow rate of the sample solution cannot be reproducibly controlled as an airgap will allow expansion and compression depending on the size of the gap. This reduces or eliminates precise control of the sample solution through the constriction, thus reducing or eliminating the reproducibility of the transfection outcome.

The pre-filling is accomplished similar to the way that air bubbles are eliminated in a medical syringe. That is, sample solution, with or without cells, is added to the container 102 by drawing the plunger 110 away from the distal end, the assembly 101 is inverted so that air escapes, and the plunger 110 is depressed toward the distal end of the container 102. The user interface 120 can then be used to accelerate the movement of the plunger 110 to the desired “forth” velocity which moves the plunger 110 away from the constriction 108. This in turn draws the sample solution 154 containing cells and molecules to be transfected through the constriction 108 into the container 102. The plunger 110 is moved initially to the off-set position 124. The user interface 120 can then be used to decelerate and stop the movement of the plunger 110 and at a second position 123 at which a desired volume of sample solution 154 has moved through the constriction 108. The user interface 120 can then to be used to accelerate the movement of the plunger 110 to the desired “back” velocity which moves the plunger 110 toward the constriction 108 and pushes the sample solution 154 through the constriction 108. The user interface 120 can then be used to decelerate and stop the movement of the plunger 110 at a third position 124 offset from the starting position 122. The back and forth movements of the plunger 110 are then repeated for a desired number of cycles. After completion of the desired number of cycles, the plunger 110 is moved from the third position 124 back to the original starting position 122. Between each inflow (movement from position 124 to 123) and outflow (movement from position 123 to 124), the plunger position is held for a period of time (125a and 125b).

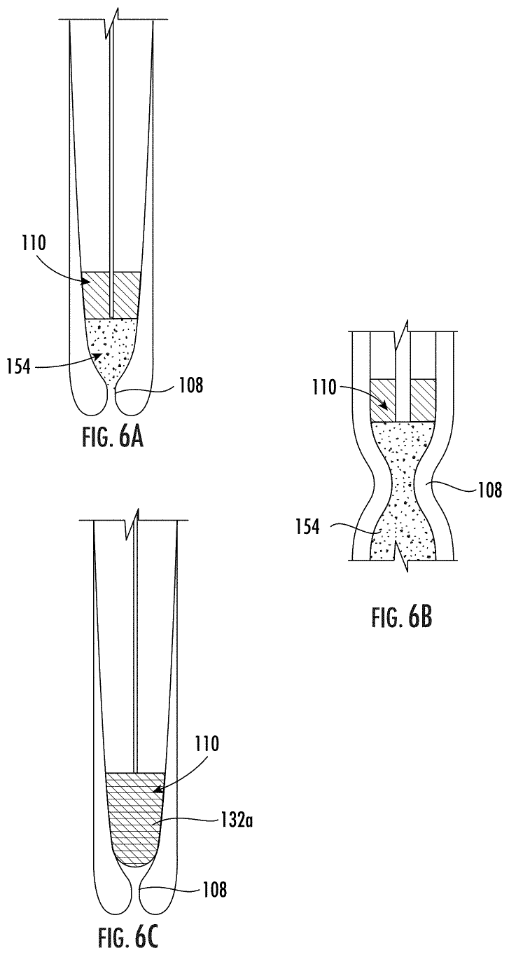

FIGS. 6A-C show alternative designs for pre-filling the container 102. FIGS. 6A and 6B include a sealable vacuum tube extending through the plunger 110, which can be used to draw the sample solution 154 into the container 102 from a reservoir 115 (not shown). FIG. 6C shows an alternative design having a non-compressible material 132a adhered to the plunger 110. The alternative in FIG. 6C is formed by first placing the non-compressible material 132a in a highly viscous, deformable phase, onto the surface of the plunger 110. The plunger 110 is then pushed toward the constriction 108 until it “fits” the constriction. The plunger is then heated to harden the viscous material into the non-compressible material 132a.

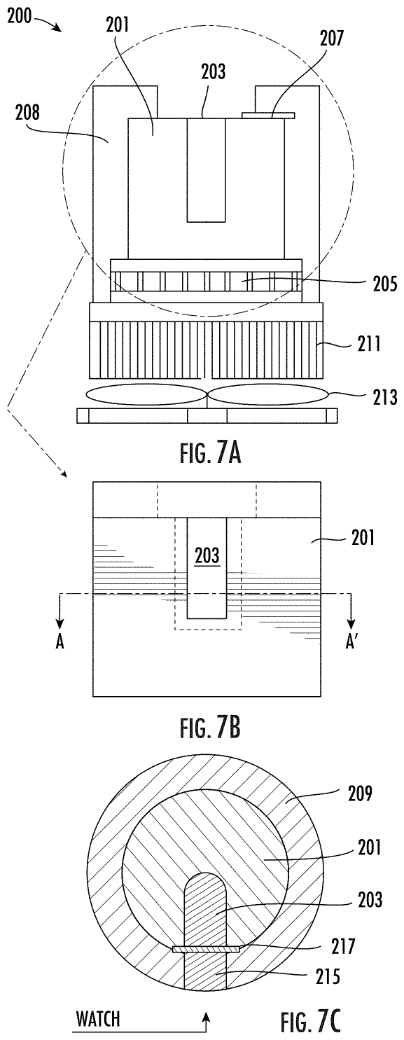

It is contemplated by this disclosure that the system 100 could include a heat unit for maintaining the sample solution 154 at a desired temperature. For example, Peltier devices offer a practical way of temperature adjustment and control at low thermal energy balances, specifically if operational cycles below and above the room temperature are required. FIG. 7A shows a sample fluid temperature control unit 200 for small sample fluid volumes: a thermal block 201 surrounded by a thermal insulator 209 with a small container volume can be used. A hole 203 is drilled in a copper cylinder of an appropriate size to fit the container 102 of the systems described herein. Close to the sample opening, on the opposite side of the Peltier device 205, a temperature sensor 207 is attached to the thermal block 201 to measure the temperature which is applied to the sample container and to provide feedback for a temperature control loop. Below the Peltier device 205 there is a heat exchanger 211 and a ventilator 213.

FIG. 7B is a front view of the temperature block 201 and sample hole 203.

FIG. 7C shows an embodiment having the sample hole 203 extended like a slot towards the periphery of the thermal block 201 and thermal insulator 209. There is a cut in the thermal insulator 215 for observing the transfection process. The thermal block 201 is closed with a glued glass plate 217 or a segment of a cut glass tube.

The block is mounted on the front panel of the transfection instrument below the container-plunger assembly in such a way so that the container 102 can be inserted into the hole 203.

It is further contemplated that the system 100 could include multiple arms 140 that operate multiple plungers 110, each plunger 110 located inside a container 102. The multiple containers 102 can be different sizes in order to accommodate various sample solution volumes. The multiple arms 140 can be connected to multiple motors 114 in order to accommodate various transfection parameters, such as different plunger speeds and different number of cycles through the respective constrictions.

It is further contemplated that the system 100 could include an optical sensor (not shown) optionally connected to a user interface.

FIG. 23 shows an embodiment including a container (e.g. capillary) and an impeller pump. In this embodiment, fluid (i.e. the sample solution together with the cells and the to be transfected molecules) is moved through a rotating part (impeller) rather than being moved by a linear motion of a plunger. An impeller pump contains a rotating component that drives the sample fluid along the pump casing. The pump casing is connected to the containers with the constriction; thus the fluid is driven from the pump through the containers and through the constriction. This allows a continuous flow of the sample fluid through the container constrictions, thus enabling a transfection device design which can handle large sample fluid volumes. Open, as well as semi-closed or closed impellers can be used, as well as axial or radial designs, using propeller, paddle, or turbine concepts.

In the devices, instruments and systems discloses herein, the plunger is in direct contact with the sample solution. The plunger can consist of a solid or liquid part, or a combination thereof, as long as none of the parts are compressible and don't mix with the sample solution and are in immediate contact with the sample solution.

In the impeller design of the pump, there is no air or compressible interface between the part of the pump which moves the sample solution and the sample solution itself.

The pump design can be a direct lift, or a displacement, or a gravity pump design. Other designs include reciprocating (the plunger moves back and forth) or rotary (for example impeller) designs, resulting in either positive displacement or centrifugal or axial-flow pumps. These designs include micro pump designs as well as internal gear, screw, shuttle block, flexible or sliding or rotating vane, circumferential piston, flexible impeller, helical twisted roots (e.g. Wendelkolben pump) or liquid ring pumps; piston or plunger or diaphragm, or rope or chain, or gear or screw or peristaltic or triplex-style plunger pump designs.

Plungers can range from stabilized ferrofluids to oils in immediate contact with solid plungers as listed above. In addition to the plungers described herein, anything that causes fluid to flow can be used as a plunger.

Key for success is that the plunger consists of a solid or liquid part, or a combination thereof, as long as none of the parts are compressible and don't mix with the sample solution and are in immediate contact with the sample solution.

### Kits

Kits for performing transfections are disclosed. In some embodiments, the kits include the container/plunger assemblies as described herein. In other embodiments, the kits include the microfluidic devices described herein. In some embodiments, the kits include a buffer or media, which can be provided in a separate vial or can be provided contained within the container/plunger assemblies or within the microfluidic devices. In some embodiments, the buffer or media includes cells or cell-like bodies.

In some embodiments, the kits further include instructions for use in accordance with the methods of this disclosure. In some embodiments, these instructions comprise a description of how to perform transfections according to any of the methods described herein. In general, the instructions include information on reagent types (e.g., buffer and/or media), amounts and concentrations, concentrations of cells or cell-like bodies, plunger positions, plunger speeds including acceleration and deceleration speeds and plunger hold times.

Methods Methods for introducing molecules or compositions in a solution into cells or cell-like bodies are disclosed.

In some embodiments, the molecules or compositions are in a solution together with the cells or cell-like bodies. In some embodiments, the solution, or a sample thereof, is loaded into a container as described herein having a constriction section. The solution, or a sample thereof, is passed through the constriction at least one time.

When performing the methods of some embodiments, the plunger(s) can be in direct contact with the sample solution.

Without intending to be bound by theory, it is believed that the transfection process described herein triggers the generation of gas and vacuum spheres which provokes endocytosis, resulting in transfection of the molecules or compositions contained in the solution, or a sample thereof. In some embodiments, the spheres that are generated are about 0.1 nm to about 100 μm. In other embodiments, the spheres that are generated are about 1 μm to about 10 μm. In other embodiments, the spheres that are generated are about 1 μm, about 5 μm, about 10 μm, about 15 μm, about 20 μm, about 25 μm, about 30 μm, about 35 μm, about 40 μm, about 45 μm, about 5 μm, about 55 μm, about 60 μm, about 65 μm, about 70 μm, about 75 μm, about 80 μm, about 85 μm, about 90 μm, about 100 μm, about 110 μm, about 120 μm, about 130 μm, about 140 μm or, about 150 μm. While spheres may be generated due to the plunger movement, in some embodiments gases (i.e., gaseous spheres) or solid materials (i.e., solid spheres) may be added during the sample loading to change the viscosity or flow pattern or other parameters of the sample solution. Representative examples of gaseous spheres include, but are not limited to, those created by adding oxygen, nitrogen or carbon dioxide. Representative examples of solid spheres include, but are not limited to, inert organic or inorganic materials such as, glass beads, latex beads, polymer beads, sugar particles, salt particles, cellulose particles, polymer particles, lipid vehicles, liposome vehicles and inert cells. In some embodiments, biologically compatible polymers can be used for the particles or beads. Representative examples of polymers that can be used for the particles or beads include, but are not limited to, polypropylene, polyethylene, polyurethanes, polycaprolactone (PCL), poly(propylene fumarate) (PPF), poly(lactide-co-glycolide) (PLGA), polylactide (PLA), poly(glycolic acid) (PGA), poly(ethylene glycol) (PEG) and collagen.

The nucleation of gas spheres and the density of gas spheres can be controlled by the roughness of the inner surface of the containers or channels as well as by the partial pressures of gases in the transfection solutions. Arithmetic average roughness can range from 1 nm to 10 μm, and more specifically from 10 nm to 1 μm. The gas partial pressure can range from 1000 Pascal to 200,000 Pascal, and more specifically from 10,000 Pascal to 120,000 Pascal of the transfection solutions.

In some embodiments, the cells to which the molecules or compositions are being introduced (i.e., the cells being transfected) are prokaryotic or eukaryotic cells. Representative examples of prokaryotic cells include bacteria, cyanobacteria and archaea. Representative examples of eukaryotic cells include animal cells, plant cells, protists and fungi. In some embodiments, the cells to which the molecules or compositions are being introduced (i.e., the cells being transfected) are animal cells including epithelial cells, endothelial cells, fibroblasts, basal cells, adipocytes, keratocytes, chondrocytes, hematopoietic cells including red blood cells, erythrocytes reticulocytes, or platelets, stem cells (including hematopoietic stem cells, embryonic stem cells or induced pluripotent stem cells), spleen cells, kidney cells, pancreas cells, liver cells, neuron cells, glial cells, muscle cells, smooth muscle cells, heart cells, lung cells, ocular cells, bone marrow cells, gametes (oocytes and sperm cells), fetal cord blood cells, progenitor cells, tumor cells, peripheral blood mononuclear cells, immune cells (including, but not limited to, leukocyte cells, lymphocyte cells, T cells, B cells, natural killer (NK) cells, dendritic cells (DC), natural killer T (NKT) cells, mast cells, granulocytes, innate lymphoid cells, monocytes, macrophages, basophils, eosinophils or neutrophils).

In some embodiments, the cells include physiologically inactive cells, for example inhibited, UV-inactivated, enucleated, anucleate or heat-killed. In certain embodiments, the cells include non-reproducing cells or synthetic cells having an artificial membrane. In certain embodiments, the cells include healthy cells, infected cells or diseased cells.

In some embodiments, the cells are primary cells. In other embodiments, the cells are cultured. In some embodiments, the cells are synchronized so that the majority of cells are in the same cell cycle phase when used in the methods described herein.

In some embodiments, the cells are autologous cells. Autologous cells are cells from one subject serving as both donor and recipient, i.e. cells are isolated from a subject, modified or treated ex vivo, and re-introduced into the same subject. In other embodiments, the cells are allogenic cells. Allogenic cells are cells isolated from a donor subject, modified or treated ex vivo, and introduced to a recipient subject who is different than the donor subject.

In some embodiments, the molecules or compositions are introduced into cell-like bodies. Representative examples of cell-like bodies include, but are not limited to, exosomes, vesicles, organelles, membrane-bound sub-cellular vesicles and cell-derived or synthetically-derived membrane-bound vesicles or sub-cellular vesicles.

As discussed above, in some embodiments, the cells are passed through a constriction that is 2 to 10 times larger than the diameter of the cells. Typically, animal cells have cell diameters ranging from about 4.5 to 120 μm. Representative cells and their average diameters are listed in Table 1.

TABLE 1

| Cell Type | Diameter (μm) |
|---|---|
| NIH 3T3 | 15 |
| Algae (various) | 7-9 |
| CHO | 14-17 |
| Cos-7 | 15 |
| Epithelia | 14-15 |
| HEK293 | 11-15 |
| HeLa | 12-14 |
| HepG2 | 12 |
| HT-29 | 11 |
| B cells | 6-11 |
| Human embryonic stem cells | 9-12 |
| HUVEC | 14-15 |
| Jurkat | 13 |
| K562 | 22 |
| MCF7 | 15-17 |
| MDCK | 13-15 |
| Mouse embryonic stem cells | 5-13 |
| Mesenchymal stem cell | 15-16 |
| PBMCs | 7-12 |
| PC12 | 9-13 |
| Primary astrocytes | 7 |
| Rat whole blood | 4.6 |
| Rat dorsal root ganglion cells | 7 |
| Red blood cells | 5-7 |
| Rat neural stem cells | 11-13 |
| SF9 | 13 |
| SH-SY5Y | 12 |
| Splenocytes | 7-9 |
| U266 | 12 |
| U87-human glioblastoma cell line | 12-14 |
| Yeast-Pichia Pastoris | 5 |
| Yeast-S. cerevisiae | 6 |
| Human oocytes | 120 |

In some embodiments, the cells are suspended in a cell culture medium or a buffer solution at physiological pH (pH of 7.4) prior to transfection. Representative examples of buffered solutions include phosphate buffered saline (PBS) and cell culture media such as M199, RPMI-1640, DMEM or IMDM. Other physiologically compatible buffer solutions and cell culture media are known in the art, and can be appropriately selected based on the combination of the cell type being transfected and the material being introduced into the cells.

In various embodiments, up to about 10 million cells are contained in a 1000 solution. In some embodiments, about 1 million cells are contained in a 1000 solution. In other embodiments, about 100 thousand cells are contained in a 1000 solution. The size and shape of the assemblies used in the methods described herein can be varied to accommodate sample volumes up to and exceeding litres, containing and exceeding tens of millions of cells and down to as low as sub-microliters containing one or more cells.

In various embodiments, the molecules or compositions to be introduced into cells include, but are not limited to, nucleic acids, peptides, proteins, carbohydrates, lipids, viral compounds (e.g. viruses and viral-like particles), organic and/or inorganic compounds, synthetic polymers, drugs, pharmaceutical compositions or combinations or mixtures thereof.

Representative examples of nucleic acids include deoxyribonucleic acids (DNA), ribonucleic acids (RNA), DNA/RNA hybrid molecules, and DNA or RNA with one or more modified nucleotides that increase stability or half-life of the DNA or RNA in vivo or in vitro. In some embodiments, DNA includes cDNA and methylated DNA. In some embodiments, RNA includes mRNA, tRNA, rRNA, siRNA, shRNA, PiRNA, RNAi, miRNA and dsRNA. In some embodiments, the nucleic acid is a vector, plasmid or transposon. In some embodiments, the nucleic acid is an expression vector carrying a nucleic acid that encodes a protein or peptide. In certain embodiments, the expression vector encodes an antibody, antibody fragment or chimeric antigen receptor (CAR).

A representative example of a synthetic polymer includes peptide nucleic acids (PNA). Representative examples of viral compounds include viruses and viral-like particles.

Representative examples of proteins include, but are not limited to, structural proteins (e.g., keratin), contractile proteins (e.g., actin), storage proteins (e.g., egg whites), defence proteins (e.g., antibodies), transport proteins (e.g., haemoglobin), signalling proteins (e.g., hormones) and enzyme proteins (e.g., lactose). In certain embodiments, the proteins are antibodies, antigens, hormones, enzymes or any natural. Representative examples of peptides include, but are not limited to, synthetic proteins or short natural or synthetic peptides.

Representative examples of antibodies include, but are not limited to, polyclonal antibodies, monoclonal antibodies, chimeric antibodies, humanized antibodies and human antibodies. The antibodies may be obtained from any species of animal, e.g., a human, simian, mouse, rat, rabbit, guinea pig, horse, cow, sheep, goat, pig, dog or cat. In some embodiments, the class of antibody that may be used, includes IgG₁, IgG₂, IgG₃, IgG₄, IgM, IgA₁, IgA₂, IgD and IgE antibodies. Antibodies or antibody fragments, which also may be used, include single chain antibodies, F(ab′)2 fragments, Fab fragments, Fv fragments including single-chain variable fragment (scFv), disulfide stabilized Fv fragments (dsFv), single variable region domains (dAbs), minibodies, combibodies, multivalent antibodies such as diabodies and multi-scFv, single domains from camelids such as nanobodies or engineered human equivalents, and fragments produced by an Fab expression library.

Representative examples of combinations of molecules or compositions to be introduced into cells include a mixture of protein and genetic material (i.e., nucleic acid), such as ribonucleoproteins (RNP) including gene editing components or gene editing complexes. In certain embodiments, the gene editing components or gene editing complexes include, but are not limited to, CRISPR components, such as a Cas protein or Cpf1 protein and guide RNA (gRNA), donor DNA or a CRISPR RNA (crRNA) and a trans-activating crRNA (tracrRNA). In yet other embodiments, the gene editing components or gene editing complexes include, but are not limited to, a TALEN protein, a zinc finger nuclease (ZFN), a mega nuclease or a Cre recombinase.

Representative examples of pharmaceutical compositions include, but are not limited to an anti-tumor agent, an antiviral agent, an antibacterial agent, an anti-mycobacterial agent, an anti-fungal agent, an anti-proliferative agent, a pro-apoptotic agent, an anti-migration agent, a toxin-binder, a receptor down-regulator, an internal signalling cascade disruptor and an anti-apoptotic agent.

One parameter affecting the transfection efficiency includes the amount of genetic material (i.e., nucleic acid) or protein use per transfection. In some embodiments, the amount of DNA or protein used per transfection is about 20 to 150 μg/ml. In other embodiments, the amount of DNA or protein used per transfection is about 1 to 1000 μg/ml. In other embodiments, the amount of DNA or protein used per transfection is about 1 μg/ml, about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 110 μg/ml, about 120 μg/ml, about 130 μg/ml, about 140 μg/ml, about 150 μg/ml, about 160 μg/ml, about 170 μg/ml, about 180 μg/ml, about 190 μg/ml, about 200 μg/ml, about 300 μg/ml, about 400 μg/ml, about 500 μg/ml, about 600 μg/ml, about 700 μg/ml, about 800 μg/ml, about 900 μg/ml, or about 1000 μg/ml. Another parameter affecting the transfection efficiency includes the size of the genetic material (i.e., nucleic acid) or protein used per transfection.

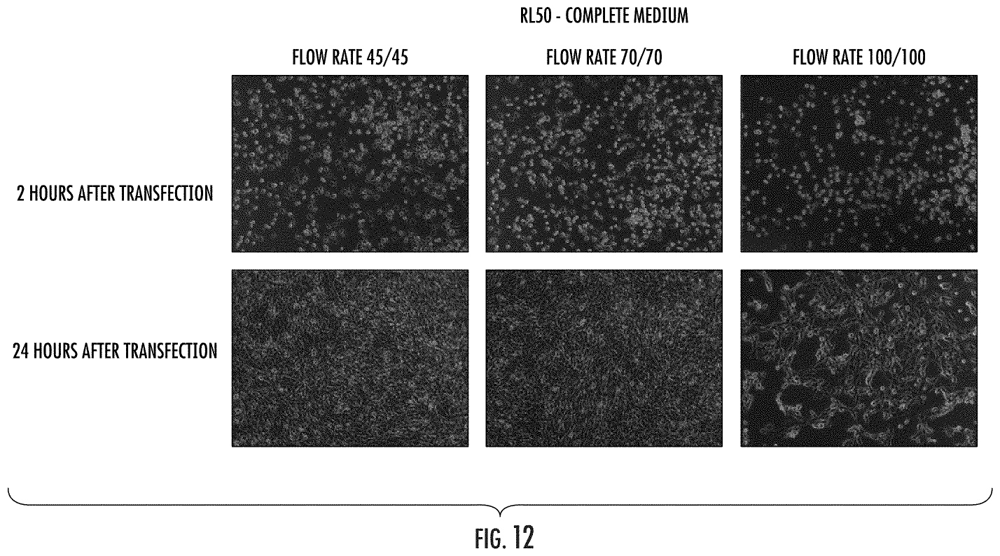

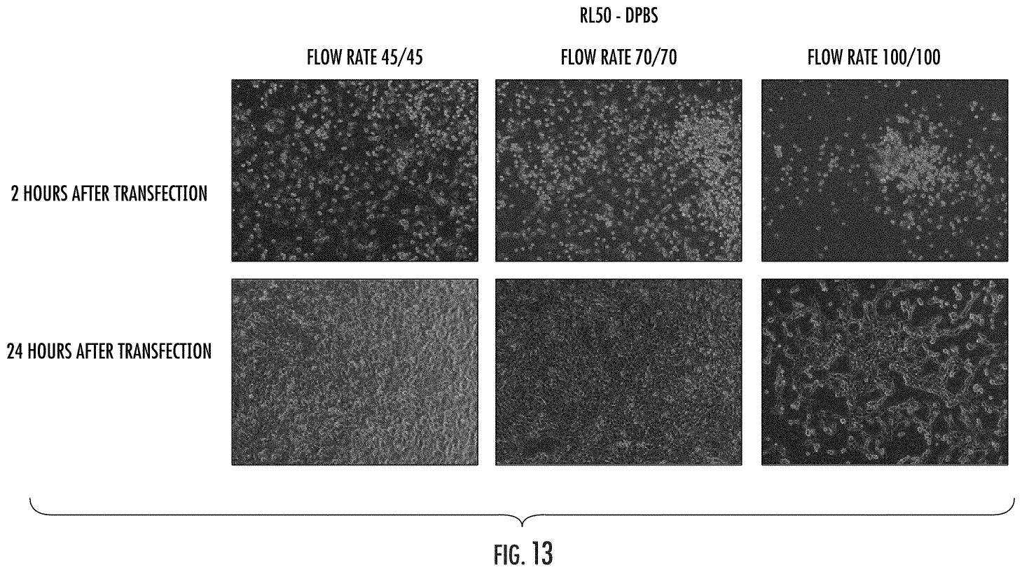

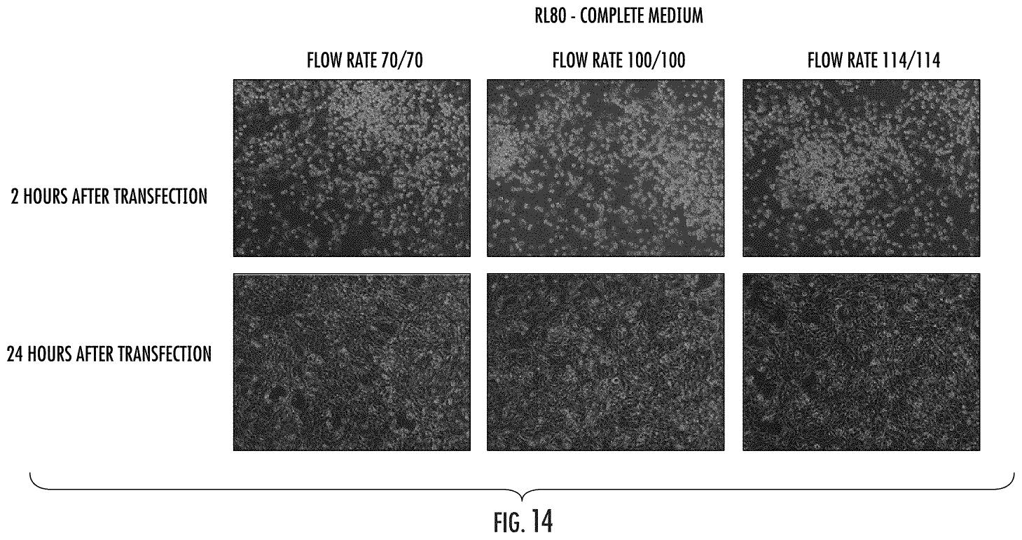

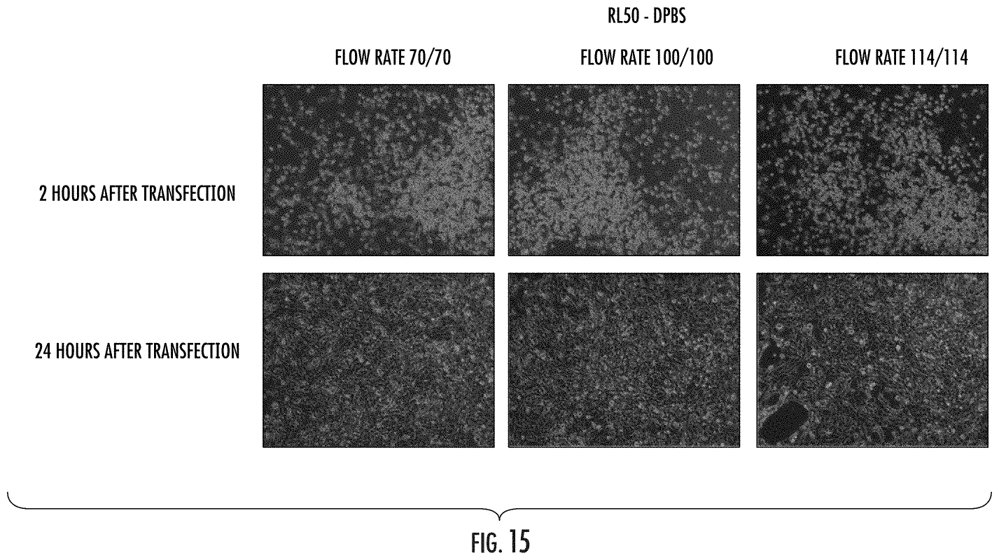

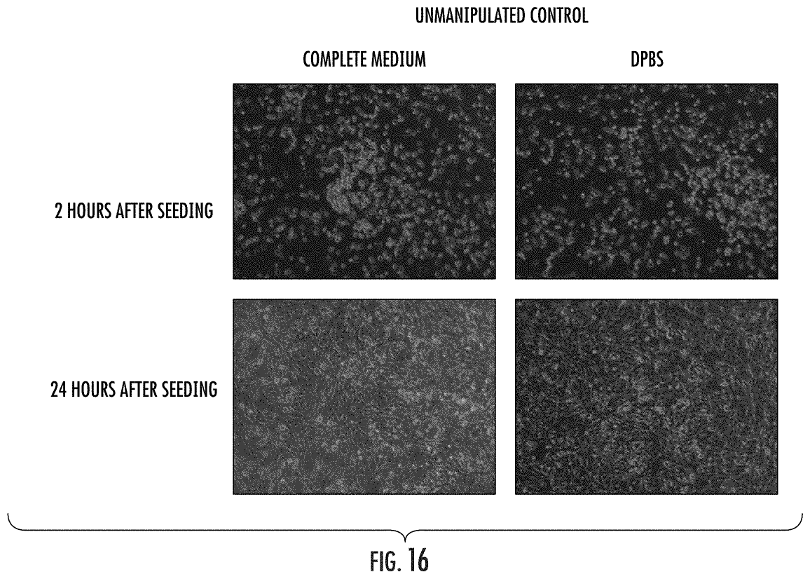

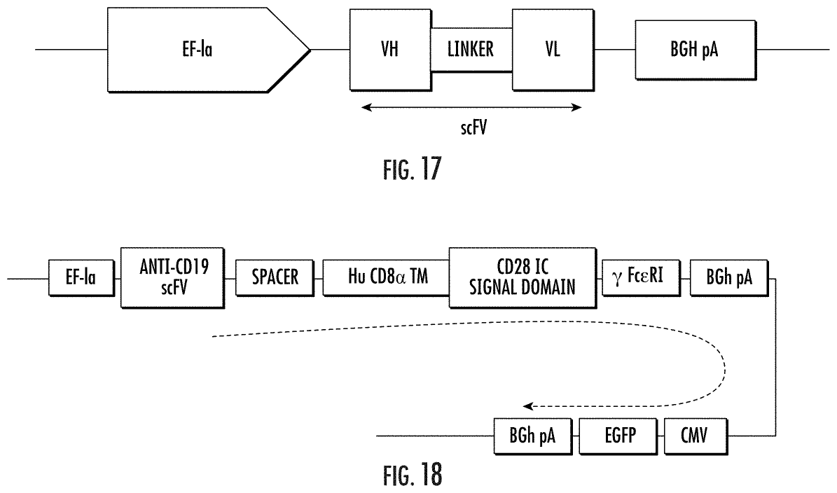

Plunger velocity, acceleration and deceleration rates and holding times also affect transfection efficiency. Example holding times can include 1-5 seconds, 5 minutes, 10 minutes or longer. In some embodiments, the rate of flow of the transfection sample is about 10 to about 1000 μl/sec. In certain embodiments, the inward and outward flow rates are the same. Representative examples of flow rates include 30/30, 40/40, 45/45, 47/47, 50/50, 60/60, 70/70, 80/80, 90/90, 100/100 and 114/114 microliters per second. In yet other embodiments, the inward and outward flow rates can differ. The flow rates can be adjusted based on various parameters including the type of cells, the size of the cells, the sizes of the container and constriction and the volume of transfection solution. The flow rate is determined by the plunger velocity. The flow rates described herein are an average flowrate because the flow rate of a solution flowing in a cylindrical tube is not uniform at a cross-sectional area, but follows a Gaussian distribution. Moreover, the flow rates in the constriction section are far faster. The flow rate across the constriction also follows a Gaussian distribution, but this distribution is far steeper than in the non-constricted sections of the container. FIGS. 12-16 show the effect of flow rates on cell survivability.

Number of flow cycles, i.e., the number of times the sample containing the cells and the molecules or compositions to be transfected passes through the constriction is another parameter affecting the transfection efficiency. One flow cycle includes one inflow step and one outflow step. Therefore, the cells pass through the constriction two times during each flow cycle. In some embodiments, the number of flow cycles is more than one cycle. In certain embodiments, the number of flow cycles is 5-25 cycles, preferably 15 cycles.

The cells and cell-like bodies modified by the transfection methods of the present disclosure (referred to herein interchangeably as “transfected cells”, “transfected cell-like bodies”, “modified cells”, “modified cell-like bodies”, “engineered cells” or “engineered cell-like bodies”) can be used in a variety of applications including treating human or animal diseases, creating replacement cells, and creating therapeutics. In addition, cells and cell-like bodies modified by the transfection methods of the present disclosure can be used in manufacturing (e.g. generating biological therapeutics), for agricultural and nutritional value improvement (e.g. genetically-modified organisms; “GMO's”) or for environmental modulation (e.g. digesting environmental toxins).

Therapeutically effective populations of engineered cells or engineered cell-like bodies prepared using the containers and methods described herein can be administered to subjects in need thereof. The number of engineered cells or engineered cell-like bodies administered to a subject will vary between wide limits, depending upon the location, type, and severity of the condition being treated, the age and condition of the individual to be treated, etc. In some embodiments, a physician may determine appropriate dosages to be used. In general, formulations containing therapeutically effective populations of engineered cells or engineered cell-like bodies are administered that contain from about 1×10⁴ to about 1×10¹⁰ engineered cells or engineered cell-like bodies. In some embodiments, the formulation containing therapeutically effective populations of engineered cells or engineered cell-like bodies are administered that contain from about 1×10⁵ to about 1×10⁹ engineered cells or engineered cell-like bodies, from about 5×10⁵ to about 5×10⁸ engineered cells or engineered cell-like bodies, or from about 1×10⁶ to about 1×10⁷ engineered cells or engineered cell-like bodies.

The formulation containing therapeutically effective populations of engineered cells or engineered cell-like bodies may be administered to a subject in need thereof in accordance with acceptable medical practice. An exemplary mode of administration is intravenous injection. Other modes include, but are not limited to, intratumoral, intradermal, subcutaneous (s.c., s.q., sub-Q, Hypo), intramuscular (Lin.), intraperitoneal (i.p.), intra-arterial, intramedullary, intracardiac, intra-articular (joint), intrasynovial (joint fluid area), intracranial (including convection-enhanced delivery), intraspinal, and intrathecal (spinal fluids). Any known device useful for parenteral injection or infusion of the formulations can be used to effect such modes of administration. Such formulations may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

One representative example of a use for the therapeutically effective populations of engineered cells or engineered cell-like bodies transfected by the containers and methods disclosed herein is for protecting a subject against an infectious agent or reducing the likelihood of a subject being infected by an infectious agent. In some embodiments, such methods include providing cell-like bodies or autologous or allogenic cells that are transfected using the systems and assemblies described herein. After transfection, the cells or cell-like bodies are infused into a subject in need of protection against an infectious agent. In some embodiments, prior to infusion, the transfected cells are optionally grown ex vivo to increase the number of cells. In some embodiments, the transfection material can be an expression vector encoding an antibody or an antibody fragment that binds to the infectious agent or to a toxic substance produced by the infectious agent.

Representative examples of infectious agents include bacteria, viruses, fungi, parasites and prions. Representative examples of toxic substances produced by infectious agents include toxins (e.g. botulinum toxin) and allergens.

Another representative example of a use for the therapeutically effective populations of engineered cells or engineered cell-like bodies transfected by the containers and methods disclosed herein is for the production of CAR-T cells for use in therapeutic treatments. In some embodiments, such methods include providing autologous or allogenic cells that are transfected using the systems and assemblies described herein. After transfection, the cells are infused into a subject in need of treatment. In some embodiments, prior to infusion, the transfected cells are grown ex vivo to increase the number of cells. In some embodiments, the transfection material is donor DNA encoding a chimeric antigen receptor that binds a tumor-associated antigen packaged in, for example, an adeno-associated viral (AAV) vector or a plasmid or provided as a DNA minicircle, as linear dsDNA or as mRNA. Genetically modifying T cells to express chimeric antigen receptors using mRNA provides a fast and economical method, however the transgene is not integrated into the host cell genome, and thus expression is rapidly diluted over the expansion of the T cells. In some embodiments, RNA transfection is used to evaluate potential toxicities or to limit the side effects of the therapy. Non-targeted integration of donor DNA (plasmid or minicircles) into the host cell genome can be accomplished by co-transfection with transposase enzymes, such as Sleeping Beauty or piggyBac. Targeted integration of donor DNA (AAV or linear dsDNA) into the host cell genome can be accomplished by co-transfection with endonuclease enzymes, such as zinc-finger, TALENs or CRISPR/Cas9.

The CAR-T cells produced by the transfection methods disclosed herein can be used for treating cancer by engineering the T cells to express a chimeric antigen receptor that binds to a tumor-associated antigen. Other CAR-T cell strategies are known in the art including universal CARs, which involve an antibody-based molecule that recognizes a tumor-associated antigen and is modified to express a “tag” and a universal CAR-T cell that recognizes and binds to the “tag”. Another strategy is a split-CAR system named SUPRA CAR, which combines zipCAR-T cells containing an extracellular leucine zipper with a scFv domain fused to a second leucine zipper (zipFv). Representative examples of cancers treated with CAR-T cells include, but are not limited to, blood cancers, such as non-Hodgkin lymphoma and acute lymphoblastic leukemia. CAR-T cells can also be used to treat solid tumors.

Another representative example of a use for the therapeutically effective populations of engineered cells or engineered cell-like bodies transfected by the methods disclosed herein is in gene therapy applications. Gene therapy generally falls into three categories: i) replacing a defective or mal-adaptive gene (e.g. curing or at least ameliorating the symptoms of a monogenic or polygenic disease or disorder), ii) altering or killing an aberrant cells (e.g. cancerous cells or cells infected with a virus such as HIV) and iii) inducing production of a therapeutic protein (e.g., treating diabetes by promoting production and secretion of insulin by cells or treating hepatitis C by promoting production and secretion of interferon by cells). In some embodiments, the transfection material includes donor DNA encoding an appropriate transgene to i) replace a defective or mal-adaptive gene associated with a disease or disorder, ii) alter or kill an aberrant cell or iii) induce production of a therapeutic protein. Similar to genetically engineering CAR-T cells as discussed above, the transfection material can further contain proteins or genetic material encoding proteins that function to integrate the transgene into the host genome. Representative examples include, but are not limited to, transposase enzymes (such as Sleeping Beauty and piggyBac), endonuclease enzymes (such as zinc-finger, TALENs and CRISPR/Cas9), genetic material (i.e., nucleic acid) encoding transposase enzymes or genetic material (i.e., nucleic acid) encoding endonuclease enzymes.

Representative examples of diseases or conditions that could be treated using gene therapy facilitated by the transfection methods disclosed herein include, but are not limited to, monogenic disorders, polygenic disorders, neurological diseases, cardiovascular diseases, autoimmune diseases, inflammatory diseases, cancers, ocular diseases and infectious diseases.

Representative examples of monogenic and polygenic disorders that can be treated with genetically engineered cells produced using the transfection methods disclosed herein include, but are not limited to, sickle cell anemia, severe combined immunodeficiency (ADA-SCID/X-SCID), cystic fibrosis, hemophilia, Duchenne muscular dystrophy, familial hypercholesterolemia, alpha-1 antitrypsin deficiency, chronic granulomatus disorder, Fanconi anemia, Gaucher disease, Leber's congenital amaurosis, phenylketonuria, thalassemia, oculocutaneous albinism, Huntington's disease, myotonic dystrophy, neurofibromatosis, polycystic kidney disease, hypophosphatemic rickets, Rett's syndrome, nonobstructive spermatogenic failure, fragile X syndrome, Friedreich's ataxia, spinocerebellar ataxias, Van der Woude syndrome, cancer, heart disease, diabetes, schizophrenia, Alzheimer's disease, Parkinson's disease, 22q11.2 deletion syndrome, Angelman syndrome, Canavan disease, Charcot-Marie-Tooth disease, color blindness, Cri du chat, Down syndrome, haemochromatosis, Klinefelter syndrome, Prader-Willi syndrome, spinal muscular atrophy, Tay-Sachs disease and Turner syndrome.

Additional representative examples of monogenic and polygenic disorders that can be treated with genetically engineered cells produced using the transfection methods disclosed herein include, but are not limited to, 1p36 deletion syndrome, 18p deletion syndrome, 21-hydroxylase deficiency, 22q11.2 deletion syndrome, Alpha 1-antitrypsin deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Aicardi-Goutières syndrome, Albinism, Alexander disease, alkaptonuria, Alport syndrome, Alternating hemiplegia of childhood, Amyotrophic lateral sclerosis-Frontotemporal dementia, Alström syndrome, Alzheimer's disease, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Björnstad syndrome, Bloom syndrome, Birt-Hogg-Dubé syndrome, Brody myopathy, Brunner syndrome, CADASIL syndrome, CARASIL syndrome, Chronic granulomatous disorder, Campomelic dysplasia, Canavan disease, Carpenter Syndrome, Cerebral dysgenesis-neuropathy-ichthyosis-keratoderma syndrome (SEDNIK), Cystic fibrosis, Charcot-Marie-Tooth disease, CHARGE syndrome, Chédiak-Higashi syndrome, Cleidocranial dysostosis, Cockayne syndrome, Coffin-Lowry syndrome, Cohen syndrome, collagenopathy (types II and XI), Color blindness, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Darier's disease, Dent's disease (Genetic hypercalciuria), Denys-Drash syndrome, De Grouchy syndrome, Down Syndrome, Di George's syndrome, Distal hereditary motor neuropathies, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich's ataxia, G6PD deficiency, Galactosemia, Gaucher disease, Gerstmann-Sträussler-Scheinker syndrome, Gillespie syndrome, Glutaric aciduria (type I and type 2), GRACILE syndrome, Chronic Granulomatus disorder, Griscelli syndrome, Hailey-Hailey disease, Harlequin type ichthyosis, Hereditary Haemochromatosis, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington's disease, Hunter syndrome, Hurler syndrome, Hutchinson-Gilford progeria syndrome, Familial Hypercholesterolemia, Hyperlysinemia, Primary Hyperoxaluria, Hyperphenylalaninemia, Hypoalphalipoproteinemia (Tangier disease), Hypochondrogenesis, Hypochondroplasia, Hypophosphatemic rickets, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Keloid disorder, Klinefelter syndrome, Kniest dysplasia, Kosaki overgrowth syndrome, Krabbe disease, Kufor-Rakeb syndrome, LCAT deficiency, Leber's congenital amaurosis, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Familial Mediterranean fever, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy (Duchenne and Becker type), Myostatin-related muscle hypertrophy, Myotonic dystrophy, Natowicz syndrome, Neurofibromatosis type I, Neurofibromatosis type II, Niemann-Pick disease, Nonketotic hyperglycinemia, Nonobstructuive spermatogenic failure, Nonsyndromic deafness, Noonan syndrome, Norman-Roberts syndrome, Oculocutaneous albinism, Ogden syndrome, Omenn syndrome, Osteogenesis imperfecta, Pantothenate kinase-associated neurodegeneration, Parkinson's disease, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thalassemia, Thanatophoric dysplasia, Treacher Collins syndrome, Tuberous sclerosis complex (TSC), Turner syndrome, Usher syndrome, Van der Woude syndrome, Variegate porphyria, von Hippel-Lindau disease, Waardenburg syndrome, Weissenbacher-Zweymüller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (a.k.a., triple X syndrome), XXXX syndrome (a.k.a., 48, XXXX), XXXXX syndrome (a.k.a., 49, XXXXX), XYY syndrome (a.k.a., 47,XYY), Zellweger syndrome, cancer, heart disease, diabetes, and schizophrenia.

Representative examples of types of cancers that can be treated with the CAR-T cells or other genetically engineered cells produced using the transfection methods disclosed herein include, but are not limited to, carcinomas derived from epithelial cells (including cancers developing in the breast, prostate, lung, pancreas and colon), sarcomas arising from connective tissue (i.e. bone, cartilage, fat and nerve tissues), lymphomas and leukemia arising from cells that make blood, germ cell tumors derived from pluripotent cells and most often presenting in the testicle or ovary, and blastomas derived from immature “precursor cells or embryonic tissue.

Representative examples of cancers that can be treated with the CAR-T cells or other genetically engineered cells produced using the transfection methods disclosed herein include, but are not limited to, Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer (islet cell), Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter (transitional cell cancer), Prostate cancer, Testicular cancer, Gestational trophoblastic tumor, Ureter and renal pelvis (transitional cell cancer), Urethral cancer, Uterine sarcoma, Vaginal cancer, Vulvar cancer, Wilms tumor, Esophageal cancer, Head and neck cancer, Head and neck squamous cell carcinoma, Nasopharyngeal carcinoma, Oral cancer, Oropharyngeal cancer, Paranasal sinus and nasal cavity cancer, Pharyngeal cancer, Salivary gland cancer, Hypopharyngeal cancer, Acute biphenotypic leukemia, Acute eosinophilic leukemia, Acute lymphoblastic leukemia, Acute myeloid leukemia, Acute myeloid dendritic cell leukemia, AIDS-related lymphoma, Anaplastic large cell lymphoma, Angioimmunoblastic T-cell lymphoma, B-cell prolymphocytic leukemia, Burkitt's lymphoma, Chronic lymphocytic leukemia, Chronic myelogenous leukemia, Cutaneous T-cell lymphoma, Diffuse large B-cell lymphoma, Follicular lymphoma, Hairy cell leukemia, Hepatosplenic T-cell lymphoma, Hodgkin's lymphoma, Hairy cell leukemia, Intravascular large B-cell lymphoma, Large granular lymphocytic leukemia, Lymphoplasmacytic lymphoma, Lymphomatoid granulomatosis, Mantle cell lymphoma, Marginal zone B-cell lymphoma, Mast cell leukemia, Mediastinal large B cell lymphoma, Multiple myeloma/plasma cell neoplasm, Myelodysplastic syndromes, Mucosa-associated lymphoid tissue lymphoma, Mycosis fungoides, Nodal marginal zone B cell lymphoma, Non-Hodgkin lymphoma, Precursor B lymphoblastic leukemia, Primary central nervous system lymphoma, Primary cutaneous follicular lymphoma, Primary cutaneous immunocytoma, Primary effusion lymphoma, Plasmablastic lymphoma, Sézary syndrome, Splenic marginal zone lymphoma, T-cell prolymphocytic leukemia, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor and Liposarcoma.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed. Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used in the specification and claims, for the purposes of describing and defining the disclosure, the terms “about” and “substantially” are used to represent the inherent degree of uncertainty that may be attributed to any quantitative comparison, value, measurement, or other representation. The terms “about” and “substantially” are also used herein to represent the degree by which the quantitative representation may vary from a stated reference without resulting in a change in the basic function of the subject matter at issue. “Comprise”, “include”, and/or plural forms of each are open ended and include the listed parts and can include additional parts that are not listed. “And/or” is open-ended and includes one or more of the listed parts and combinations of the listed parts.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Method of Manufacturing Transfection Containers with Constriction

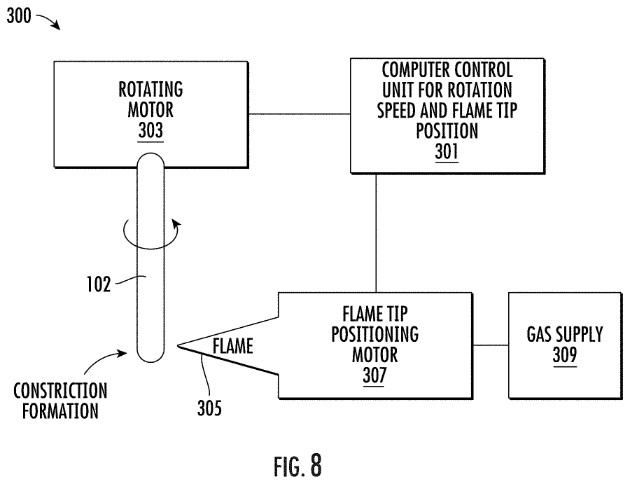

FIG. 8 is a diagram of the system 300 for fabricating the constrictions in transfection containers 102. A programmable computer 301 controls a motor 303 with a holder to mount a container 102. The motor rotates the container along its longitudinal axis. The computer also controls the position and size of a micro flame 305 so that the tip of the flame is precisely positioned by a motor 307 along the container to heat the container until the desired constriction diameter and shape is achieved. The constriction diameter was measured using a microscope and the diameter data was communicated to the computer and the flaming and rotating process was stopped when the desired outcome was achieved (e.g., a distal end constriction as in FIG. 2A; or an hourglass design as in FIG. 2B).

In the examples below, glass capillaries were used as the container with constriction.

### Example 2: DNA Transfection

DNA transfections were performed using NIH/3T3 cells line (mouse; cell diameter about 15 μm) or HeLa cell line (human; cell diameter about 12-14 μm). Plasmid vectors, 4.7 kb, expressing a green fluorescent protein (GFP) under the control of a Cytomegalovirus promoter were used for DNA transfections. All transfections were performed with the cells dispersed in a physiological buffer solution (Dulbecco's Modified Essential Medium (DMEM), Medium 199 or Dulbecco's phosphate buffered saline) at a cell density of 1 to 10 million cells per milliliter of transfection mix. As used herein, “transfection mix” and “transfection solution” are used interchangeably and includes buffer solution, the molecules to be transfected and anything else that might be contained in the solution to increase the efficiency of the transfection. About 100 microliters of the transfection mix was used per transfection reaction. The amount of DNA used per transfection varied between 20 to 150 micrograms per milliliter.

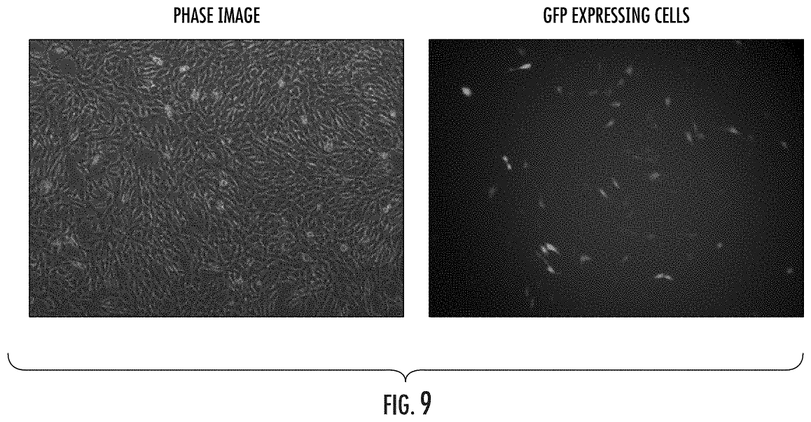

The results of DNA transfection were evaluated after 24 hours (FIG. 9) after the transfections were performed by detecting the cytoplasmic expression of GFP with fluorescence microscopy. GFP expression levels varied from weak to very strong expression. The expression strength varies between cells because of inherent heterogeneity of the cell population. Expression of GFP indicated the successful introduction of plasmid DNA into the cell cytoplasm, transport of the plasmid into the nucleus, subsequent RNA transcription in the nucleus and translation of RNA resulting in cytoplasmic GFP.

Estimated DNA transfection efficiency indicated that the efficiency depended on several parameters. A relative transfection efficiency for a 4.7 kb plasmid DNA is presented in Table 2. Approximately 250,000 NIH/3T3 cells were resuspended in 100 μl transfection solution containing 15 μg of DNA and transfections were performed using 15 cycles at the various flow rates indicated in Table 2. Various dimensions of capillaries at various flow rates were analyzed. “Relative” efficiencies are indicated with a “+” sign. The efficiency ranges from + to ++++, where “+” represents relative transfection efficiency of the cells of approximately 5-14%, “++” represents relative transfection efficiency of the cells of approximately 15-29%, “+++” represents relative transfection efficiency of the cells of approximately 30-59%, and “++++” represents relative transfection efficiency of the cells of approximately 60-100%, This is based on the number of cells expressing GFP as compared between the parameters used. For this study, the 80RL capillary at a flow rate of 114/114 (inward/outward) yielded the highest DNA transfection efficiency for the 4.7 kb plasmid.

TABLE 2
DNA transfection efficiency for 4.7 kb pAcGFP-1 plasmid

| | Flow velocity (rate) | | | Relative |
|---|---|---|---|---|
| Capillary | μL/sec | | Relative cell | transfection |
| used | Inward | Outward | survival | efficiency |
| 50 RL | 30 | 30 | ++++ | + |
| 50 RL | 40 | 40 | ++++ | + |
| 50 RL | 50 | 50 | +++ | ++ |
| 50 RL | 60 | 60 | +++ | +++ |
| 50 RL | 70 | 70 | ++ | ++ |
| 50 RL | 80 | 80 | + | + |
| 70 RL | 50 | 50 | ++++ | + |
| 70 RL | 60 | 60 | ++++ | + |
| 70 RL | 70 | 70 | ++++ | + |
| 70 RL | 80 | 80 | +++ | ++ |
| 70 RL | 90 | 90 | +++ | ++ |
| 70 RL | 100 | 100 | +++ | +++ |
| 70 RL | 114 | 114 | ++ | ++ |
| 80 RL | 80 | 80 | ++++ | + |
| 80 RL | 90 | 90 | ++++ | ++ |
| 80 RL | 100 | 100 | ++++ | ++ |
| 80 RL | 114 | 114 | +++ | ++++ |
| 100 RL | 80 | 80 | ++++ | + |
| 100 RL | 90 | 90 | ++++ | + |
| 100 RL | 100 | 100 | +++ | ++ |
| 100 RL | 114 | 114 | +++ | ++ |

Column 1 in Table 2 reflects the capillary (i.e. container) used. Values 50 μm, 70 μm, 80 μm and 100 μm indicate the smallest diameter at the constriction. “RL” indicates that the reduction of the inner diameter of the container down to the minimum constriction diameter was done over a longer stretch of distance (about 1 mm to about 25 mm) versus “RS” where the reduction of the inner diameter of the container down to the minimum diameter was accomplished over a shorter length (i.e., it was formed more abruptly; about 0.1 mm to 1 mm). Thus, the distance from the beginning of the reduction of the inner diameter to the minimum diameter is between about 0.1 mm to 25 mm.

### Example 3: Protein Transfection

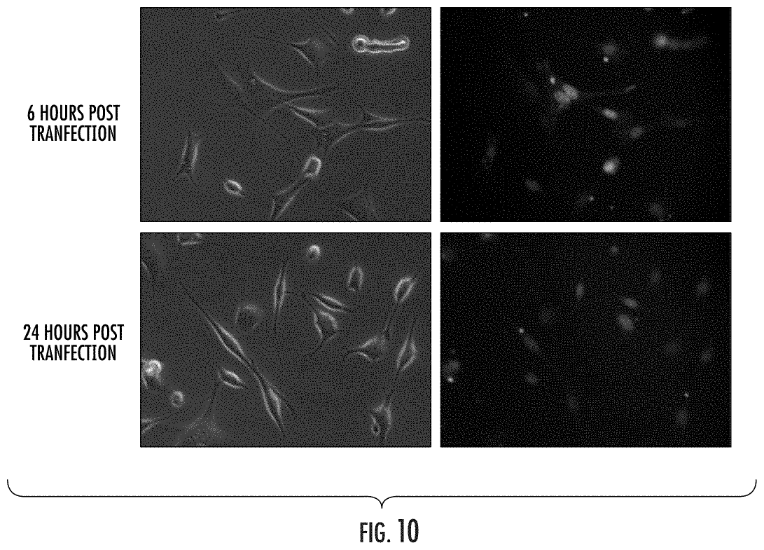

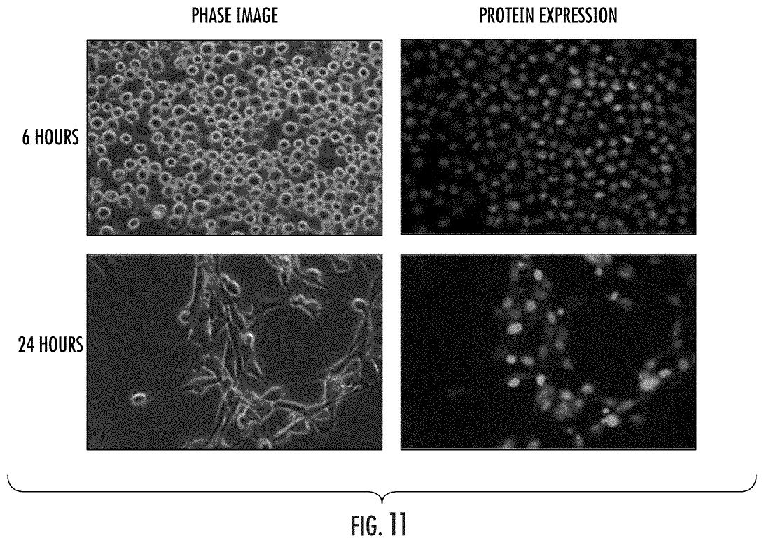

In the protein transfection study using a 50RL capillary with Alexa Fluor 488 conjugated to a nuclear localized 22 kDa protein, both the cell survival rate and transfection efficiency exceeded 95% (FIGS. 10 and 11). FIG. 10 shows NIH/3T3 cells 6 and 24 hours post transfection with 8 μg protein using a flow rate of 30/30 microliters per second for 15 cycles. FIG. 11 shows HeLa cells 6 and 24 hours post transfection with 8 μg protein using a flow rate of 30/30 microliters per second for 15 cycles.

### Example 4: Effect of Capillary Constriction Size on Cell Survival

Glass capillaries of various constrictions were used to flow the cells for 15 cycles without the addition of DNA, RNA or protein in the transfection mix. Results are presented in FIGS. 12-16. Unmanipulated cells, that is, cells not passed through the capillaries were used as control experiments. It was observed that narrower constrictions and higher flow rates resulted in a progressive loss of cells.

Cell survival/cell loss due to transfection was quantified by enumerating cell numbers using a flow cytometer. NIH-3T3 cells that were not passed through a capillary were used as controls. For the experimental group, the same number of cells (approximately 100,000) were suspended in M199 culture media or Dulbecco's phosphate buffered saline to pass through two capillary sizes for 15 cycles at 4 different flow rates as shown in Table 3. The smaller capillary, 50RL, exhibited higher levels of cell loss at all flow rates compared to the 80RL capillary.

TABLE 3
Effect of flow rate on cell survival/cell loss resulting from transfection

| | Number of cells detected in sample | | |
|---|---|---|---|
| Flow rates | No capillary | 50RL | 80RL |
| (μL/sec) | (control) | Capillary | Capillary |
| No flow | 94,737 | N/A | N/A |
| 47/47 | N/A | 61,941 | N/A |
| 70/70 | N/A | 30,856 | 93,537 |
| 90/90 | N/A | 21,264 | 86,164 |
| 100/100 | N/A | 10,307 | 81,038 |
| 114/114 | N/A | N/A | 71,889 |

### Example 5: Self-Production of scFV Biotherapeutic to Provide Therapeutic Protection Against BoNT/a Intoxication

A mammalian expression vector was designed that contains the EF-1a promoter functionally linked to a cDNA gene encoding a single chain FV (scFV) that strongly binds and neutralized the BoNT/A (botulinum neurotoxin serotype A) binding domain (H_{C}) and a BGH poly-adenylation sequence. The remaining sequences in the vector do not contain sufficient viral sequences to allow replication within the recipient cell. An example vector is pcDNA3 with CMV promoter replaced with Ef-1a promotor and in which the selectable marker for Neomycin has been deleted. This combination enables the high expression of the scFV in a wide variety of cells while avoiding any sequences that promote replication in mammalian cells.

Whole blood will be extracted from Balb/c mice into tubes containing citrate, phosphate, dextrose and adenine (CPDA) to inhibit clotting while also stabilizing the cells and the blood pooled. The whole blood will be layered over a Ficoll-Paque gradient and spun to concentrate the mononuclear white blood cells (WBC). The isolated WBC will be washed twice in PBS and resuspended with 2.5×10⁵ cells in 50 μl of PBS. 100 μg of the expression vector encoding the anti-BoNT/A scFV will be added to the cells and preincubated for 5-10 minutes at RT. The cell/DNA mixture will then be subjected to 15-25 cycles of positive and negative fluid pressure and allowed to recover briefly.

The cells will be plated in media for 21 days with media and samples taken daily to measure the quantities of anti-BoNT/A scFV being produced by the transfected primary cells. As needed, fresh media will be added.

Because the cells are terminally differentiated and have not received any DNA that will alter normal cell life stages, over time, the cells succumb to normal cell senescence and die. As the transfected cells senesce, the concentration of scFV in the media will decrease and finally disappear.

### Example 6: Therapeutic Protection Against BoNT/a Intoxication in Mice

The experiment described in Example 5 will be repeated with the following changes. As above, the blood is extracted from Balb/c mice and the WBCs will be isolated and transfected as described. Instead of plating the cells, the transfected WBCs will be slowly infused into additional Balb/c mice. After several days, the mice will be injected with varying doses of BoNT/A ranging from sub-lethal through lethal. The mice will be followed for development of BoNT/A intoxication and death to determine the protective effects of the transfected expression vector and biotherapeutic protein. Control mice, also injected with anti-BoNT/A scFV transfected WBCs, will be tested over time for the amount of anti BoNT/A scFV produced by the transfected WBCs over time.

### Example 7: Creation of Anti-CD19 CAR-T Cells for the Destruction of Malignant B Cells

A mammalian expression vector was designed that contains the EF-1a promoter functionally linked to a cDNA gene encoding an anti-CD19 CAR construct and a BGH poly-adenylation sequence. The anti-CD19 CAR construct is similar to that described by Dr. Kochendenfer in US2017/0107286 A1. In addition to the anti-CD19 scFV, the CAR construct contains an extracellular spacer, a transmembrane region of human CD8alpha, the intracellular T-cell signaling domains derived from human CD28 and the gamma chain of Fc epsilon RI. The remaining sequences in the vector do not contain sufficient viral sequences to allow replication within the recipient cell. An example vector is pcDNA3 in which the selectable marker for Neomycin has been replaced with functional cassette for GFP. This combination enables the high expression of the CAR construct in a wide variety of cells while avoiding any sequences that promote replication in mammalian cells. The GFP will allow for internal expression of green fluorescence protein that can be used to follow successfully transfected cells.

Whole human blood will be obtained by standard blood collection into blood collection bags containing citrate, phosphate, dextrose and adenine (CPDA) to inhibit clotting while also stabilizing the cells and pooled. The red blood cells will be lysed by spinning down the whole blood and discarding the supernatant. The pellet will be resuspended in RBC lysis solution and after 10 minutes, diluted in PBS, spun down and washed in PBS. Anti-CD4 or anti-CD8 antibodies conjugated to magnetic beads will be added to the white blood cells and dripped through a magnetized column. After washing, the column will be demagnetized and the CD4 and CD8 T cells collected.

The CD4 or CD8 T cells will be washed twice in PBS and resuspended as 2.5×10⁵ cells in 50 ul of PBS. 100 ug of the anti-CD19 CAR expression vector will be added to the cells and preincubated for 5-10 minutes at RT. The cell/DNA mixture will then be subjected to 15-25 cycles of positive and negative fluid pressure and allowed to recover briefly.

After transfection, the cells will be cultured with anti-CD3/anti-CD28 beads to trigger the development of activated CAR T cells. At various times, samples will be taken for anti-CD3 anti-CAR construct and GFP FACS screening.

### Example 8: Cell Killing Assay Using Transfected T Cells

To measure the ability of the transfected T cells to kill the human target cell line, Raji (ATCC CCL86) that have high levels of surface expression of human CD19 expression will be obtained. Raji cells are used as a surrogate for malignant B cells. The Raji cells will be first dyed with CellTracker Red (Thermofisher) and washed to remove all excess dye. The Raji cells and CAR expressing T cells will be combined in different concentrations and placed in culture. At various times over the next 36 hours, samples will be analyzed by FACScan looking for the disappearance of the Raji cells by following the disappearance of the Red Cell Tracker dye. The presence of the CAR T cells can be followed by anti-CD3 and anti-CD19-CAR antibodies and GFP. When the time-frame with the highest rate of Raji cell destruction is determined, the experiment will be repeated but followed by confocal microscope with measurements obtained every few minutes.

### Example 9: Creation of Repaired Liver Hepatocytes

A DNA vector was designed that contains a germline region sequence of the human SERPINA1 gene. To add a c-Myc tag, the cDNA sequence for c-Myc is inserted between the final codon of the SERPINA1 gene and its stop codon. This will allow for the creation of a SERPINA1 protein that can be observed in cells that have successfully undergone CRISPR targeted gene replacement.

### Example 10: Targeted Gene Replacement in Human Hepatocytes

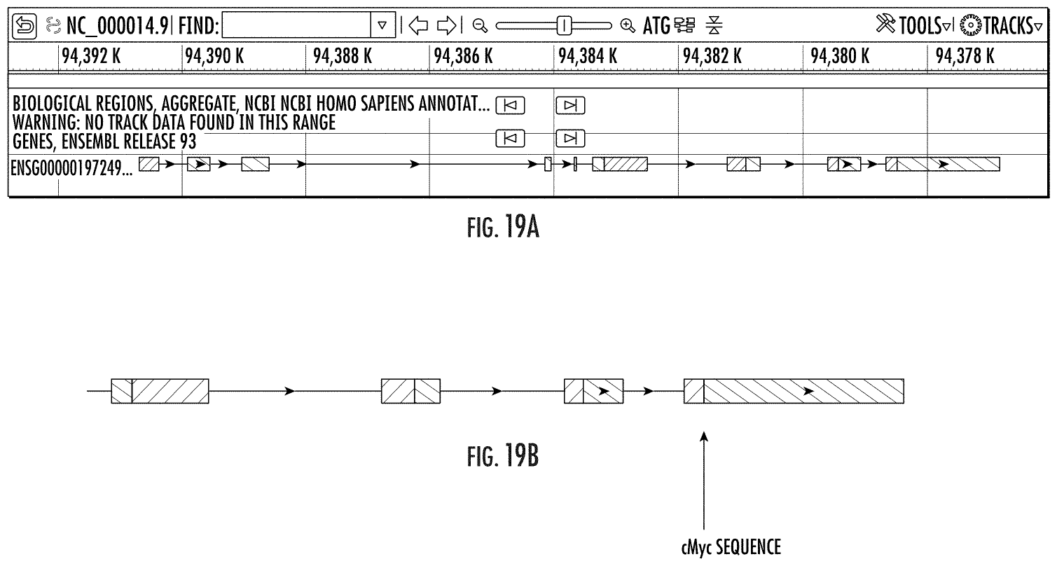

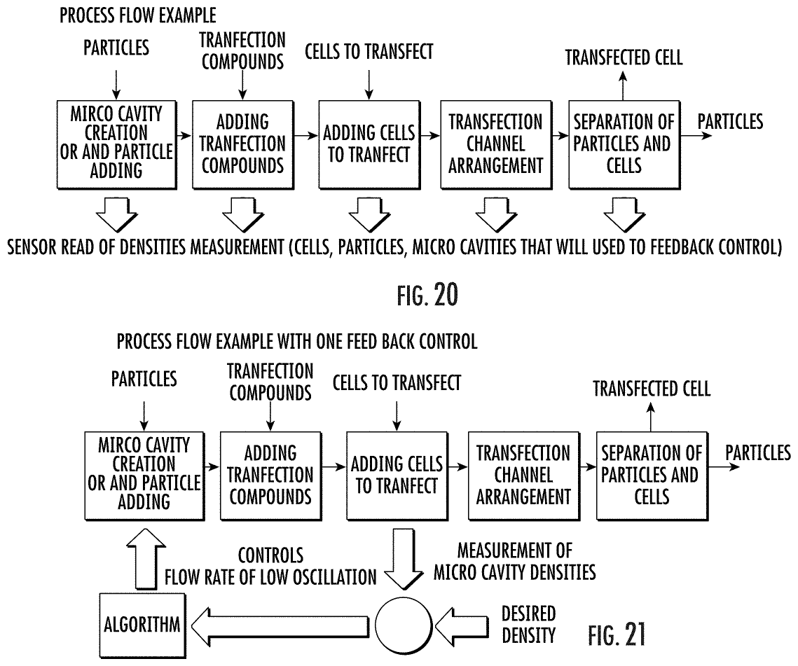

As hepatocytes from AAT enzyme deficient patients cannot be easily obtained, a surrogate experiment using CRISPR technology to replace a normal SERPINA 1 gene with a tagged version will be conducted instead. For this experiment, the human neonatal hepatocyte cell line ATCC CRL 4021 will be obtained from ATCC and expanded in culture. As it is an adherent cell line, a non-enzyme cell dissociation reagent (Thermo-Fisher) will be used to create single cell preps. The hepatocytes will be washed twice in PBS and resuspended as 2.5×10⁵ cells in 50 μl of PBS. Different amounts of the guide RNA, a combination of Cas-9 binding and a 20-mer chosen from the SERPINA1 genomic DNA (FIG. 19A) and different amounts of the S. pyogenes Cas9 (SpCas9) (Polypus) will be combined with the cells and preincubated for 5-10 minutes at RT. A control transfection containing only the tagged SERPINA 1 gene (FIG. 19B) will be used to visualize the amount of random DNA insertion (vs. the targeted gene replacement). The cell/RNA/protein or control cell/DNA mixture will then be subjected to 15-25 cycles of positive and negative fluid pressure and allowed to recover briefly before plating. Over the subsequent days, samples of the transfected cells will be harvested and used to make protein preps. A non-transfected hepatocyte cell sample will be used as control. The protein preps will be separated on an acrylamide gel and transferred to membrane. Following standard Western techniques, the membrane will first be visualized with the anti-alpha-1 Antitrypsin antibody (Thermofisher) to determine the total amount of AAT enzyme, both c-Myc tagged and untagged and then visualized with anti-c-Myc antibody. The ratio of total to tagged AAT enzyme will be used to determine which experimental combination was most effective at causing the targeted gene replacement into the human hepatocytes.

### Example 11: Transfection of Human T-Cells

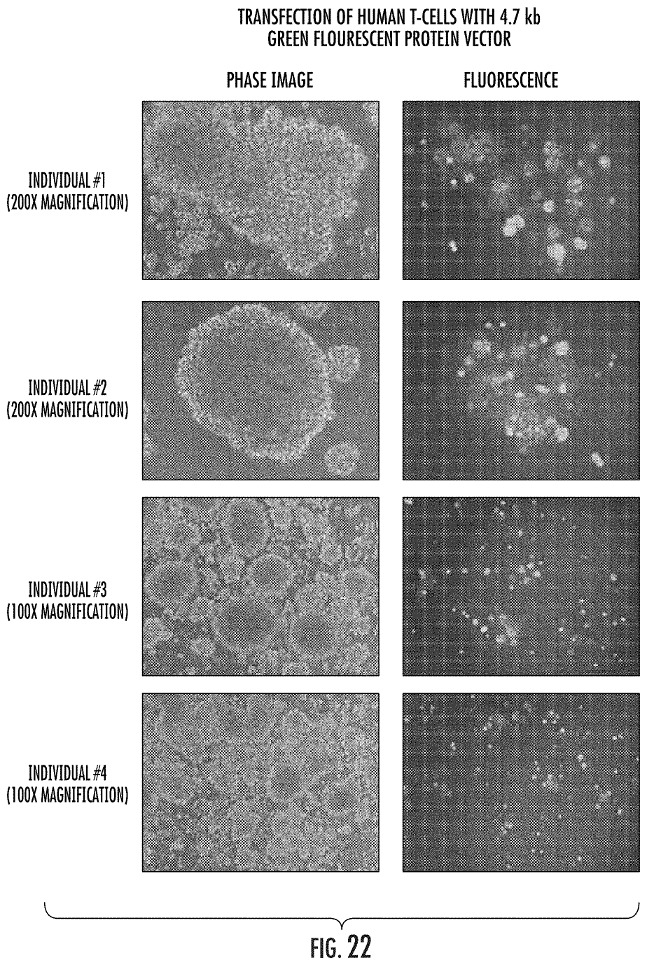

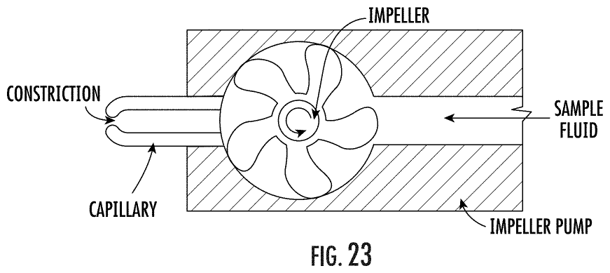

Isolated human T-cells were obtained from 4 different individuals and placed in culture with T cell culture media. The T-cells were harvested and transfected with 15 μg pAcGFP vector (4.7 kb) in complete medium using a 70RL capillary with 15 cycles at a flow rate of 80/80 microliters per second. After transfection, the T cells were returned to culture and at different time points, observed for the appearance of GFP. The results shown in FIG. 22 indicate successful transfection based on expression of GFP.

### Example 12: Computational Fluid Dynamics (CFD) Modeling

Under the inventors' direction, scientists at the company IMPACT modelled the fluid flow in a constriction and found that shear forces/stresses cause cell distortions when they flow through the constriction (and not Bernoulli pressures), and therefore the way in which cells are deformed can be influenced or controlled by changing the viscosity of the buffer solution encompassing the cells. Thus, different buffer systems can be tailored to both the type of molecule(s) being introduced into cells and the type of cells being transfected.

IMPACT has performed CFD modeling a capillary geometry having a minimum inner diameter (I.D.) of 50 μm. The model was based on the case where a plunger pushes the liquid through the capillary at a flow rate of 50 μL/s. In this case, liquid viscosity was assumed to be water-like.

In the table below, IMPACT's CFD predictions are compared with those of Timm Tanzeglock (“A Novel Lobed Taylor-Couette Bioreactor for the Cultivation of Shear Sensitive Cells and Tissues”, DSc thesis presented to ETH, Zurich, 2008). D_capillary is the minimum I.D., Q is the steady-state flow rate, U_capillary is the average velocity in the minimum I.D. portion of the capillary, tau_wall is the wall shear stress range over the middle 400 um of the capillary, tau_ext is the extensional stress range over the 200 um entrance region of the capillary, tau_IS is the hydrodynamic stress due to turbulence, and delta P is the predicted pressure drop across the capillary.

| D_capillary | Q | U_capillary | ταυ_wall | ταυ_ext | ταυ_IS | δελτα P |
|---|---|---|---|---|---|---|
| (um) | (uL/s) | (m/s) | (Pa) | (Pa) | (Pa) | (Pa) |
| 50 | 50 | 25.5 | 300-9000 | 20-400 | 2400-17000 | 280400 |
| 200 | 59 | 1.9 | 72 | 105 | — | 10950 |
| 200 | 119 | 3.8 | 145 | 237 | 185 | 29670 |
| 200 | 203 | 6.5 | 247 | 444 | 591 | 71870 |
| 200 | 267 | 8.5 | 324 | 611 | 1010 | 116700 |
| 200 | 360 | 11.5 | 524 | 836 | 1720 | 206500 |
| 200 | 450 | 14.3 | 869 | 1029 | 2590 | 316200 |

IMPACT's CFD predictions are presented in the first row of the above table, while Tanzeglock's predictions occupy the remaining rows. Under the conditions modeled by IMPACT, wall shear stress is of sufficiently high magnitude to generate “pores” in cell membranes. The wall shear stress (tau_wall) is not strongly affected by entrance and exit effects. Thus, the effects of shear stress on a cell membrane can be regulated by varying the length of minimum capillary I.D., thus varying exposure time to shear stress. Increasing viscosity increases shear stress at equal flow rate. Extensional stress (tau_ext) occurs primarily at the entrance to the capillary. Lengthening the capillary would have little, if any, effect on extensional stress. Increasing viscosity increases extensional stress. Turbulent stress (tau_IS) occurs primarily as flow exits the capillary. It is also not affected by capillary length. Increasing viscosity decreases turbulent stresses.

Pressure drops of the magnitudes predicted by IMPACT or Tanzeglock are not expected to affect cell membranes. Tanzeglock included a CFD prediction of pressure drop as a convenient means of validating his CFD model using an easy parameter to measure, but did not consider effects of pressure drop on cells. Since cells contain an incompressible fluid and are suspended in an incompressible fluid, there is no apparent mechanism to exert stress on the cell membrane due to pressure changes in the suspending liquid of the order of a few atmospheres. (The delta P value predicted by IMPACT corresponds to −3 atm). Effects on cells with cell walls have been observed at extremely high hydrostatic pressures (>4000 atm), as reported by Hartmann et al (“Mechanical stresses in cellular structures under high hydrostatic pressure”, Innovative Food Science and Emerging Technologies, 7:1-12, 2006).

Pressure forces can affect cells in flow experiments when pressure gradients occur at a length scale comparable to the cell size. This is the case with turbulence in the intertial subrange—i.e. when the Kolmogorov eddy size is smaller than the cells. This factor is summed up by tau_IS. Such microscopic pressure gradients are caused by turbulence and are not related to the Bernoulli Effect.

Under the set of conditions that IMPACT studied, the instant CFD analysis indicates that hydrodynamic stresses associated with turbulent eddies smaller than the 15 um cells being studied are most likely to affect cell membranes. In addition, the effects of shear and extensional stresses, may also contribute to the observed effects. The effect of pressure drop due to the Bernoulli Effect on suspended cells is very unlikely to have an effect on the cell membrane.

### Example 13: Rapid Protection for Frontline Personnel from New Pandemic Outbreak

Late in 2019 a novel coronavirus, SARS-Cov-19, started an international pandemic and a global response. The full genome sequence was published early in 2020 enabling the race for a vaccine. Even with the relaxation of MANY safety rules, the best hope is that by the beginning of 2021 ONLY about 1 million vaccine doses will be ready (the US population alone is 330 million). Meanwhile medical staff, first responders and many patients are getting sick and are dying. The crisis is especially acute in medical and military facilities but it is also causing severe economic disruptions, including food and critical supplies. It is anticipated that the crisis will continue until 2022 and possibly beyond.

New infectious agents are developing all the time (i.e., SARS, EBOLA) but in the past they were confined to small regions. With globalization, we now know how fast they can spread. We need new methods of treatments that can be created and deployed rapidly to our first line defenders and responders. Once the people who keep us safe are protected, we can protect everyone else.

Vaccines are overwhelmingly critical at stopping diseases, but they take years to create. Even when deployed, effective immunity takes weeks to become effective in the recipients. The vaccines trigger many other components of an immune system response. One method includes blocking infection by triggering the immune system to develop B cells that can make antibodies that block the process. In the case of SARS-Cov-19, the antibodies must prevent the spike protein of the virus from attaching to the Angiotensin-converting enzyme 2 protein (ACE2) on lung and other cells, and thus prevent the virus from getting into the cells and infecting them. However, developing this immunity of the recipients takes weeks to months after receiving a vaccine. In a medical crisis, even if we have a vaccine ready to give, first line defenders and responders will die during the time which the vaccine is inducing immunity.

Protective antibodies can be created and stored in vials for up to a year but making new ones takes several years to create, manufacture and deliver where needed. This is done by finding examples of protective antibodies and then, in the lab, manufacture synthetic antibodies in mammalian cells (e.g. Chinese Hamster Ovary (CHO) cells). Although creating the new genes can be accomplished in weeks, setting up the manufacturing process takes 1-2+ years. As a stop-gap measure, antibodies are being collected from people who recovered from SARS-Cov-19 and are provided to the sickest patients. But supplies are variable and it is impossible to ensure sterility.

TransCytos' AntibodyProcess (TAbP):

TransCytos' TAbP steps are: (a) obtaining Ab DNA (b) harvesting B cells (10-50 ml of blood) from recipient (i.e. medical staff, first responders, patients, military personnel) (c) using at least one of TransCytos' assemblies, devices, systems, kits and methods, transfecting the B cells with the DNA of protective, synthetic antibodies (such as single chain variable fragment/scFV) (d) returning the transfected B cells to the recipient so that within hours, the transfected B cells will produce protective antibodies and continue to produce protection for several weeks.

Most importantly, the TransCytos cell modification (“transfection”) process uses a non-viral technology, unlike existing human therapies that require viral transfection techniques. When using viruses, the treatment can only be used once and cannot be used in immune-compromised patients.

Therefore, the TransCytos TAbP process offers a significant advantage: because the TransCytos TAbP process uses a non-viral transfection step, it enables the recipients to receive repeated treatments.

### Example 14: Functionality Study/Kill Assay Results

### Cell Types Used for Analysis

Effector cells: human T cells, which were modified (transfected) with our technology using a CAR T vector and unmodified (control) T cells.

Target cells: Raji cells (ATCC Cat #CCL86), expressing CD19 on their cell surface were used as target cells in this experiment. The target cells were modified at TransCytos to stably express Red fluorescent protein (RFP) and flow sorted for high RFP expressing cells. The RFP expressing Raji cells were used as target cells for ease of quantification by flow analysis.

Vector used: A Chimeric Antigen Receptor T cell (CAR T) plasmid vector expressing anti CD19 chimeric receptor under the control of CMV promoter and green fluorescent protein (GFP) marker under the control of an Internal Ribosome Entry Site (RES).

### Transfection and Kill Assay

Day 1—Human T cells were transfected with the CAR T vector using the TransCytos method.

Day 2—GFP expressing T cells expressing chimeric antigen were purified by flow sorting. Purified CAR T cells or unmodified T cells (control cells) were mixed with Raji-RFP cells at a ratio of 1:3 (T cells to Raji-RFP cells) and incubated for about 18 hours for cell-cell interaction and killing. Raji-RFP cells only was included as a second control.

Day 3—Cells were analyzed by flow analysis to measure the number of high expressing RFP cells (live cells) and weak RFP expressing cells (compromised/dead cells) to determine the ratio of live:dead target cells.

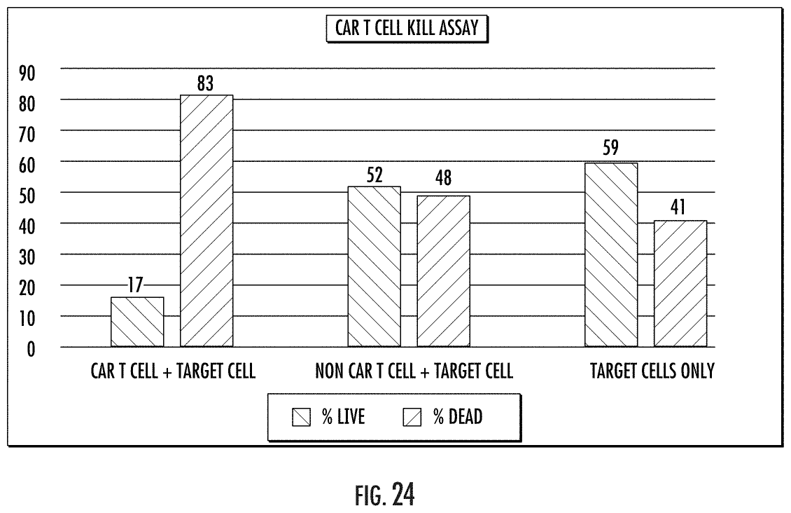

Results are presented in FIG. 24, which shows that the primary human T-cells transfected show the same functionality as the virally transfected T-cells. That is, they recognize attack and kill cancer cells.

### Result Interpretation:

CAR T cell+Target cell analysis indicated that the CAR T cells killed 83% of the target cells in approximately 18 to 20 hours of incubation.

Control T cells plus target cells showed a ratio of 52% live versus 48% dead cells (average of 3 reactions).

Target cell only control showed 59% live versus 41% dead (average of 3 reactions).

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

### EQUIVALENTS

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

1. An assembly for introducing molecules in a solution into cells or cell-like bodies comprising:
a rigid container having a first inner diameter or cross-sectional area at a proximal end thereof and inner and outer walls extending between a distal and proximal end;
a plunger insertable into the container at the proximal end; and
at least one constriction of only the inner wall proximal to the distal end or at least one constriction of the inner and the outer walls proximal to the distal end;
wherein the at least one constriction has a second inner diameter or cross-sectional area that is smaller than the container first inner diameter or cross-sectional area and the plunger is axially movable along the container.

2. The assembly of claim 1, wherein the container includes ripples that protrude away from an interior wall of the container.

3. The assembly of claim 1, wherein the constriction has a diameter that is 1.2 to 100 times larger than a diameter of the cells or cell-like bodies.

4. The assembly of claim 1, wherein the constriction has a diameter that is 2 to 10 times larger than a diameter of the cells or cell-like bodies.

5. The assembly of claim 1, wherein the plunger comprises a rod having a distal and proximal end wherein the distal end of the plunger comprises a conical or cylindrical tip and the proximal end of the plunger is configured to attach the plunger to a motorized arm.

6. The assembly of claim 1, wherein an average roughness of an inner wall of the container is 10 nm-1 μm.

7. The assembly of claim 6, the roughness is created by adsorbing cell fragments to the inner wall of the container.

8. The assembly of claim 1, wherein the container comprises a plurality of constrictions.

9. The assembly of claim 1, wherein the constriction forms a flow path having a length of a 0.2-10 mm.

10. The assembly of claim 1, wherein the container comprises a removable insert comprising a plurality of constrictions.

11. The assembly of claim 8, wherein the plurality of constrictions has a same inner diameter or cross-sectional area, a different inner diameter or cross-sectional area or combinations thereof.

12. The assembly of claim 10, wherein the plurality of constrictions has a same inner diameter or cross-sectional area, a different inner diameter or cross-sectional area or combinations thereof.

13. An assembly for introducing molecules in a solution into cells or cell-like bodies comprising:
a rigid container having inner and outer walls extending between a distal and proximal end, the outer and inner walls narrow to form a constriction in a central portion of the container between the distal and proximal ends;
a plunger movably disposed in the container near the proximal end; and
the constriction having a diameter that is 1.2 to 100 times larger than a diameter of the cells or cell-like bodies.

14. An assembly for introducing molecules in a solution into cells or cell-like bodies comprising:
a flexible container comprising a first inner diameter or cross-sectional area and a first and second end;
at least one constriction formed by compressing at least one section of the flexible container; and
a removable plunger positioned at least at one of the first or second ends or removable plungers positioned at each of the first and second ends;
wherein the at least one constriction has a second inner diameter or cross-sectional area that is smaller than the container first inner diameter or cross-sectional area.

15. The assembly of claim 14, wherein the constriction has a diameter that is 1.2 to 100 times larger than a diameter of the cells or cell-like bodies.

16. The assembly of claim 14, wherein the plungers are axially movable along the container.

17. The assembly of claim 14, wherein the at least one constriction section is formed by at least one movable wedge or at least one movable roller.

18. The assembly of claim 14, wherein the container comprises a plurality of constrictions.

19. The assembly of claim 14, wherein the container comprises a removable insert comprising a plurality of constrictions.

20. The assembly of claim 18, wherein the plurality of constrictions has a same inner diameter or cross-sectional area, a different inner diameter or cross-sectional area or combinations thereof.

21.-97. (canceled)
